(19) United States
(12) Patent Application Publication
(10) Pub. No.: US 2018/0153504 A1
(43) Pub. Date: Jun. 7, 2018

(54) 3D ULTRASOUND IMAGING, ASSOCIATED METHODS, DEVICES, AND SYSTEMS

(71) Applicants: THE BOARD OF TRUSTEES OF THE LELAND STANFORD JUNIOR UNIVERSITY, Stanford, CA (US); DUKE UNIVERSITY, Durham, NC (US)

(72) Inventors: Carl Dean HERICKHOFF, Los Altos, CA (US); Jeremy Joseph DAHL, Palo Alto, CA (US); Joshua Seth BRODER, Cary, NC (US); Matthew Robert MORGAN, Durham, NC (US)

(21) Appl. No.: 15/735,024

(22) PCT Filed: Jun. 8, 2016
(86) PCT No.: PCT/US2016/036530

§ 371 Date: Dec. 8, 2017

Related U.S. Application Data
(60) Provisional application No. 62/172,313, filed on Jun. 8, 2015.
(60) Provisional application No. 62/204,532, filed on Aug. 13, 2015.

(51) Int. Cl. A61B 8/00; A61B 8/08

(52) U.S. Cl. CPC A61B 8/4254; A61B 8/483; A61B 8/5246; A61B 8/4472; A61B 8/466; A61B 8/085; A61B 8/0891; A61B 8/4209

(57) ABSTRACT

Methods, system, and devices that are adapted to restrict the movement of an ultrasound transducer about at least one axis or point, and tag a plurality of frames of electronic signals indicative of information received by the ultrasound transducer with information sensed by an orientation sensor. The methods, system, and devices can generate a 3D ultrasound volume image of the patient by positioning the plurality of tagged frames of electronic signals at their respective orientations relative to the axis or point.

### CROSS REFERENCE TO RELATED APPLICATIONS

This application claims the priority of U.S. Provisional Application No. 62/172,313, filed Jun. 8, 2015, and U.S. Provisional Application No. 62/204,532, filed Aug. 13, 2015, the disclosures of which are incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Ultrasound is a safe, portable, fast, and low-cost imaging modality, compared to some other imaging modalities such as magnetic resonance imaging (“MRI”) and x-ray computed tomography (“CT”). MRI machines are generally very large, and require the patient to be very still during the scan, which can take a long time, even up to several minutes. CT scanners are generally very large, and while the scanning time is relatively fast compared to MRI, they deliver a relatively high dose of ionizing radiation to the patient. Ultrasound systems are portable, lower cost, and don't deliver radiation to the patient. Some of the benefits of CT and MRI scanning are that the quality of the imaging is often better than ultrasound, the patient is in a known fixed frame of reference (e.g., lying supine on a bed translated through the scanning cylinder), and the scanning captures a complete anatomic volume image dataset, which can be visualized in any number of ways (e.g., rendered in 3D or panned through slice-by-slice along any cardinal anatomical direction) by the physician after the scanning procedure.

The image quality of some 2D ultrasound systems may be considered relatively grainy, and thus not adequate in some situations where a high quality image is required. Furthermore, because 2D ultrasound is effectively a sampling of non-standardized cross-sections of a volume of the patient, 2D ultrasound does not afford the opportunity to visualize image data in planes or volumes other than those planes originally acquired.

Systems have been developed that can use ultrasound to generate a 3D volume of a portion of the patient, but to date they are very expensive, and generally do not provide a frame of reference to orient the 3D volume with respect to the patient. The lack of a reference frame can limit the utility of the images, or result in medical errors related to incorrect interpretation of the orientation of the image with respect to the patient. Some examples include systems incorporating electromagnetic sensors or application-specific matrix-array probes.

It would be beneficial to have an easy to use, more cost-effective way of generating 3D volumes of tissue using ultrasound, wherein the 3D volumes can be viewed and analyzed in real-time, near real-time, or for subsequent review, and optionally properly oriented to the patient's frame of reference (i.e., aligned with the patient's cardinal anatomical axes). Optionally, but not required, it would also be beneficial to have systems, devices, and methods that enable 3D ultrasound volume generation using existing relatively low-end 2D ultrasound equipment, which can be important in low-resource settings, including rural areas and the developing world. An emergency department is merely an exemplary setting in which it may be beneficial to provide a fast, safe, cost-effective way of obtaining 3D ultrasound volumes using existing 2D ultrasound systems.

Optionally still, it may also be beneficial to provide ultrasound systems that can aid medical personnel in obtaining and interpreting patient data, such as by annotating or providing visual guides on 2D or 3D ultrasound images, regardless of the image reconstruction method.

### SUMMARY OF THE DISCLOSURE

One aspect of the disclosure is a method, comprising: moving an ultrasound transducer and an orientation sensor stabilized with respect to the ultrasound transducer, while restricting the movement of the ultrasound transducer about an axis or point; and tagging each of a plurality of frames of electronic signals indicative of information received by the ultrasound transducer with information sensed by the orientation sensor, relative to the axis or point, each of the plurality of frames of electronic signals indicative of information received by the ultrasound transducer representing a plane or 3D volume of information within the patient. The method can be performed without sensing a position of the transducer with a position sensor.

The method can also include generating a 3D ultrasound volume image of the patient by positioning the plurality of tagged frames of electronic signals indicative of information received by the ultrasound transducer at their respective orientations relative to the axis or point.

The method can also include, prior to acquiring the electronic signals indicative of information received by the ultrasound transducer and prior to moving the transducer while restricted about the axis or point, calibrating the orientation sensor relative to a patient reference.

In some embodiments the tagged frames of electronic signals indicative of information received by the ultrasound transducer are any of raw channel data, raw beamformed data, detected data, and 3D volumes.

In some embodiments, generating a 3D ultrasound volume image of the patient occurs real-time or near-real time with the movement of the ultrasound transducer. In some embodiments, generating a 3D ultrasound volume image of the patient does not occur real-time or near-real time with the movement of the ultrasound transducer.

In some embodiments the tagging is performed by software disposed in an ultrasound system's computing station (i.e., a housing that includes hardware and software for generating and/or processing ultrasound data). In some embodiments software for generating the 3D volume of information is also disposed in an ultrasound system's computing station. Existing ultrasound systems can thus be updated with the tagging and/or 3D volume generating software, or new ultrasound systems can be manufactured to include new software and/or hardware to carry out the methods herein.

In some embodiments communication is established between an external device and one or more ultrasound system data ports. The external device can be adapted to receive as input, from the ultrasound system, a plurality of frames of electronic signals (any type of data herein) indicative of information received by the ultrasound transducer. The software for tagging and/or 3D volume generation can be disposed on the external device. In some exemplary embodiments the external device is in communication with the ultrasound system's video out port or other data port, and the external device is adapted to receive as input 2D ultrasound image data. In some embodiments the external device is adapted to receive as input raw channel data from the ultrasound system.

In some embodiments the axis or point is a first axis or point, the method further comprising restricting movement of the transducer about a second axis or point, further comprising tagging each of a second plurality of frames of electronic signals indicative of information received by the ultrasound transducer with information sensed by the orientation sensor, relative to the second axis or point, each of the second plurality of frames of electronic signals indicative of information received by the ultrasound transducer. The method can also generate a second 3D ultrasound volume of the patient by positioning the second plurality of tagged electronic signals indicative of information received by the ultrasound transducer at their respective orientations relative to the second particular axis or point. Any number of 3D ultrasound volumes can be generated using any of the methods herein, and used in any of the suitable methods herein (e.g., in any type of combining technique).

The method can also combine a first 3D ultrasound volume and a second 3D ultrasound volume together. Combining the first and second 3D volumes can create a combined 3D volume with an extended field of view relative to the first and second 3D volumes individually. Combining the first and second 3D volumes can create a combined 3D volume with improved image quality compared to the first and second 3D volumes individually. In some embodiments restricting movement about the first axis or point and the second axis or point is performed using a single movement restrictor. In some embodiments restricting movement about the first axis or point is performed with a first movement restrictor, and wherein restricting movement about the second axis or point is performed with a second movement restrictor, optionally wherein the first and second movement restrictors are fixed relative to one another at a known orientation, optionally co-planar, angled, or perpendicular.

In some embodiments the movement is restricted due to an interface between an ultrasound probe and a movement restrictor. In some embodiments the movement restrictor is part of the ultrasound probe. In some embodiments the movement restrictor is a component separate from the probe, and can be configured to stabilize the relative positions of the ultrasound probe and movement restrictor. In some embodiments the movement restrictor is part of the patient's body. In some embodiments the movement restrictor is part of the probe user's body (e.g., fingers).

In some embodiments the transducer and orientation sensor are disposed within an ultrasound probe. In some embodiments the orientation sensor is adapted and configured to be removably secured to the ultrasound probe.

The ultrasound probes herein can be wired or wireless.

One aspect of the disclosure is a computer executable method for tagging frames of electronic signals indicative of information received by an ultrasound transducer, comprising: receiving as input a plurality of frames of electronic signals indicative of information received by the ultrasound transducer, the plurality of frames of electronic signals representing a plane or 3D volume of information within a patient, wherein the movement of the ultrasound transducer was limited about an axis or point when moved with respect to the patient; receiving as input information sensed by an orientation sensor stabilized in place with respect to the ultrasound transducer; and tagging each of the plurality of frames of electronic signals indicative of information received by the ultrasound transducer with information sensed by an orientation sensor. The computer executable method can be executed without receiving as input position information of the transducer sensed by a position sensor.

In some embodiments the computer executable method is disposed in an ultrasound system housing that includes hardware and software for generating and/or processing ultrasound data. In some embodiments the computer executable method is disposed in an external computing device adapted to be in communication with an ultrasound system housing that includes hardware and software for generating and/or processing ultrasound data.

In some embodiments receiving as input a plurality of frames of electronic signals indicative of information received by the ultrasound transducer comprises receiving as input a plurality of frames of 2D ultrasound image data, and the tagging step comprises tagging each of the plurality of frames of 2D ultrasound data with information sensed by the orientation sensor.

One aspect of the disclosure is an ultrasound system that is adapted to receive as input a plurality of frames of electronic signals indicative of information received by an ultrasound transducer, the plurality of frames of electronic signals representing a plane or 3D volume of information within a patient, wherein the movement of the ultrasound transducer was limited about an axis or point when moved with respect to the patient; receive as input information sensed by an orientation sensor stabilized in place with respect to the ultrasound transducer; and tag each of the plurality of frames of electronic signals indicative of information received by the ultrasound transducer with information sensed by the orientation sensor. The ultrasound system can be further adapted to generate a 3D volume image of the patient by positioning the plurality of tagged frames of electronic signals indicative of information received by the ultrasound transducer at their respective orientations relative to the axis or point. The ultrasound system is adapted to generate the 3D volume without receiving as input transducer position information sensed by a position sensor.

One aspect of the disclosure is an ultrasound system that is adapted to generate a 3D ultrasound volume using sensed information provided from an orientation sensor that is tagged to each of a plurality of frames of electronic signals indicative of information received by an ultrasound transducer, and without using information sensed from a position sensor. The sensed information will have been sensed by an orientation sensor in a fixed position relative to the ultrasound transducer.

One aspect of the disclosure is a 3D ultrasound volume generating system, comprising: a freehand ultrasound transducer in a fixed position relative to an orientation sensor, and not a position sensor, the system adapted to generate a 3D ultrasound volume using sensed information provided from the orientation sensor that is tagged to frames of electronic signals indicative of information received by the ultrasound transducer, and without information sensed from a position sensor.

In some embodiments the system further comprises a probe movement restrictor with at least one surface configured to interface with an ultrasound probe, to limit the movement of the ultrasound transducer about an axis or point.

One aspect of the disclosure is a computer executable method for generating a 3D volume image of a patient, comprising: receiving as input a plurality of tagged frames of electronic signals indicative of information received by the ultrasound transducer, the plurality of tagged frames of electronic signals each representing a plane or 3D volume of information within a patient, each of the received plurality of frames of electronic signals tagged with information sensed by an orientation sensor stabilized in place with respect to the ultrasound transducer, wherein the movement of the ultrasound transducer was limited about a particular axis or point when moved with respect to the patient; and generating a 3D ultrasound volume image by positioning the plurality of tagged frames of electronic signals indicative of information received by the ultrasound transducer at their respective orientations relative to the particular axis or point.

The computer executable method is adapted to be executed without receiving as input position information of the transducer sensed by a position sensor.

In some embodiments receiving as input a plurality of tagged frames of electronic signals indicative of information received by the ultrasound transducer comprises receiving as input a plurality of tagged 2D ultrasound image data, and wherein generating the 3D ultrasound volume comprises positioning the plurality of tagged 2D ultrasound image data at their respective orientations relative to the particular axis or point.

One aspect of the disclosure is a method of generating a 3D ultrasound image volume, comprising: scanning a patient's body with an ultrasound probe in a fixed position relative to an orientation sensor; sensing orientation information while moving the probe, but not sensing x-y-z position information of the probe; and generating a 3D ultrasound volume from a plurality of frames of electronic signals indicative of information received by the ultrasound transducer. The method can further include restricting the movement of the probe about an axis or point.

One aspect of the disclosure is an ultrasound imaging apparatus, comprising: an ultrasound probe in a fixed position relative to an orientation sensor; and a movement restrictor configured with at least one surface to interface with the ultrasound probe, and adapted so as to limit the movement of the ultrasound probe about an axis or point, the movement restrictor further comprising at least one surface adapted to interface with the body of a patient. In some embodiments the movement restrictor has at least a first configuration (or state) and a second configuration (or state), wherein the first configuration (or state) restricts the ultrasound probe's movement about the axis or point, and the second configuration (or state) restricts the ultrasound probe's movement about a second axis or point, optionally wherein the two axes are orthogonal, or in the same plane (but not so limited). In some embodiment the movement restrictor comprises a probe cradle with at least one surface to interface with a surface of the ultrasound probe. In some embodiments the movement restrictor further comprises an axis selector, which is adapted to be moved or reconfigured to select one of at least two axes or points for restriction of movement. In some embodiments the apparatus further comprises a second movement restrictor configured to stably interface with the movement restrictor, the second movement restrictor adapted to so as to limit the movement of the ultrasound probe about a second axis or point.

One aspect of the disclosure is a 3D ultrasound image volume generating apparatus, comprising: an ultrasound probe in a fixed position relative to an orientation sensor; a movement restrictor configured so as to restrict the movement of the ultrasound probe about a particular axis or point; a tagging module adapted to tag each of a plurality of frames of electronic signals indicative of information received by the ultrasound transducer with information sensed by the orientation sensor, relative to the particular axis or point; and a 3D volume generating module adapted to position each of the plurality of orientation tagged frames of electronic signals indicative of information received by the ultrasound transducer at respective orientations, relative to the particular axis or point, to generate a 3D image.

In some embodiments the movement restrictor is integral with the ultrasound probe.

In some embodiments the movement restrictor is configured with at least one surface to interface with a surface of the ultrasound probe so as to restrict the movement of the ultrasound probe about a particular axis or point.

In some embodiments the orientation sensor is disposed within a body of the ultrasound probe.

In some embodiments the orientation sensor is adapted to be removably secured to the ultrasound probe. The apparatus can further comprise a sensing member comprising the orientation sensor, the sensing member configured with at least one surface such that it can be secured to a proximal portion of the ultrasound probe, optionally where a probe housing meets a probe cable. In some embodiments the sensing member comprises a probe interface, the probe interface optionally having an opening with a greatest linear dimension of 10 mm-35 mm, optionally 15 mm-30 mm.

In some embodiments the apparatus does not include a position sensor.

In some embodiments the movement restrictor comprises an axis or point selector adapted so that the movement restrictor can restrict the movement of the ultrasound probe about a second axis or point.

In some embodiments the movement restrictor is configured with at least one surface such that it can be positioned on the body of a patient.

In some embodiments, the apparatus further comprises an external device in communication with an ultrasound system, the external device comprising the tagging module, and receiving as input the plurality of frames of electronic signals indicative of information received by the ultrasound transducer. The external device can also be in communication with the orientation sensor. The external device can further comprise the 3D volume generating module. The external device can be in communication with a video out port of the ultrasound system. The external device can be in communication with the ultrasound system to enable the external device to receive as input from the ultrasound system at least one of raw channel data, raw beamformed data, and detected data.

In some embodiments the apparatus further comprises a second movement restrictor configured to be stabilized with respect to the movement restrictor, the second movement restrictor configured with at least one surface to interface with the ultrasound probe so as to restrict the movement of the ultrasound probe about a second particular axis or point. The tagging module can be adapted to tag each of a plurality of frames of electronic signals indicative of information received by the ultrasound transducer with information sensed by the orientation sensor, relative to the second particular axis or point, wherein the 3D volume generating module is adapted to generate a second 3D ultrasound volume of the patient by positioning the second plurality of tagged frames of electronic signals indicative of information received by the ultrasound transducer at their respective orientations relative to the second particular axis or point. The 3D volume generating module can further be adapted to merge the 3D ultrasound volume and the second 3D ultrasound volume together.

In some embodiments the tagging module and the 3D volume generating module are disposed within an ultrasound system housing that includes hardware and software for generating and/or processing ultrasound data.

One aspect of the disclosure is a sensing member with at least one surface configured to be removably secured in a fixed position relative to an ultrasound probe, the sensing member comprising an orientation sensor and not a position sensor. In some embodiments the sensing member comprises an adhesive backing. In some embodiments the sensing member has an opening, optionally, with a largest linear dimension from 10 mm-35 mm, optionally 15 mm-30 mm. In some embodiments the sensing member comprises a deformable element configured to be deformed to allow the sensing member to be secured to the ultrasound probe. In some embodiments the sensing member is adapted for wireless communication. In some embodiments the sensing member is adapted for wired communication.

One aspect of the disclosure is an ultrasound probe movement restrictor, the movement restrictor configured to stably interface with an ultrasound probe. The movement restrictor can be adapted and configured to restrict movement of the probe about one, two, three, four, five, or even more, axes or points. In some embodiments the movement restrictor is configured to be stabilized to one more movement restrictors.

### BRIEF DESCRIPTION OF THE DRAWINGS

FIG. 1A illustrates an exemplary method of generating a 3D volume, including optional calibration.

FIG. 1B illustrates an exemplary calibration process.

FIG. 1C illustrates an exemplary tagging process.

FIG. 1D illustrates an exemplary 3D volume generation process.

FIGS. 2A and 2B illustrate exemplary restricted movement of an ultrasound probe about an axis.

FIG. 3 schematically illustrates an exemplary apparatus including an ultrasound probe, orientation sensor, and movement restrictor.

FIG. 4 is a perspective view of an exemplary apparatus, including an exemplary ultrasound probe, exemplary sensing member, and exemplary movement restrictor.

FIG. 5 illustrates generally an exemplary ultrasound probe and an exemplary sensing member.

FIGS. 6B and 6C illustrate an ultrasound probe interfaced with a merely exemplary movement restrictor that is configured to restrict movement of the probe about at least one axis.

FIGS. 6A, 6D, 6E, and 6F illustrate an ultrasound probe interfaced with the exemplary movement restrictor shown in FIGS. 6B and 6C, with the movement restrictor in a second configuration or state that restricts the movement of the probe about a second axis.

FIG. 6G illustrates an ultrasound probe interfaced with the exemplary movement restrictor shown in FIG. 6A-6F, with the probe's movement being restricted about a third axis, the third axis being in the same plane as the second axis.

FIG. 7 illustrates an exemplary method of 3D volume generation.

FIG. 8 illustrates schematically an exemplary apparatus that can be used to generate a 3D volume.

FIG. 9 illustrates schematically an exemplary apparatus that can be used to generate a 3D volume.

FIGS. 10A, 10B, 11A, 11B, 12A, 12B, 13A, and 13B illustrate images annotated with exemplary patient references (which can be generated as real-time visual aids using orientation sensor information), as well as relative positioning and/or orientation of an ultrasound probe with respect to a subject's body.

FIG. 14 illustrates an exemplary apparatus that is adapted and configured to restrict a probe's movement about a plurality of axes, which can be used to allow multiple 3D volumes to be generated.

FIGS. 15A, 15B, 15C, 15D, 15E, and 15F illustrate exemplary individual components of some exemplary movement restrictors herein.

FIG. 16 is an exemplary generated 3D volume image of the face of a 36-week fetal phantom acquired and reconstructed using methods herein and an existing ultrasound system with an ultrasound scanner and probe only capable of 2D.

FIGS. 17A, 17B, 17C, 17D, and 17E illustrate exemplary visualizations of (i.e., additional images that can be obtained from) a 3D volume generated using systems and methods herein that tag frames of electronic signals with sensed orientation information.

FIGS. 18A, 18B, 18C, 18D, and 18E illustrate exemplary visualizations of (i.e., additional images that can be obtained from) a 3D volume generated using systems and methods herein that tag frames of electronic signals with sensed orientation information.

### DETAILED DESCRIPTION

This disclosure relates generally to ultrasound imaging, and more particularly to tagging frames of electronic signals indicative of information received by an ultrasound transducer with sensed orientation information, and generating a 3D volume using the tagged frames of electronic signals. The methods herein restrict movement of the ultrasound transducer about at least one axis or point, and are capable of generating the 3D volume using information sensed from an orientation sensor, without requiring position information sensed by a position sensor (i.e., from an x-y-z sensor, such as an optical position sensor or electromagnetic field sensor).

Use of position sensors (which may also incorporate orientation sensing) with ultrasound probes for volume image generation give the advantage of allowing the ultrasound probe greater freedom of movement in space and providing precise location information about the image plane from wherever the probe may be held in contact with and in relation to the patient's body. Methods using position sensors with ultrasound probes have been proposed and investigated as early as the 1990's, but precise position determination can be difficult to achieve (and often subject to many constraints or sensitive to factors in the clinical environment, such as electromagnetic noise) and the sensors or sensing systems developed to achieve this which have been used with ultrasound probes for volume image generation—are often quite complex and may come in awkward form factors. Because of this, position-sensor-based ultrasound volume image generation methods have had limited success, and generally have not been integrated into commercial ultrasound systems and have not gained traction in the marketplace. The disclosure herein includes methods that can generate 3D ultrasound volumes without requiring the use of position sensors.

The methods herein can optionally calibrate the orientation sensor with respect to a patient's orientation and use the calibration reading(s) to properly orient at least one of the 2D image data and the 3D volume with respect to the patient's cardinal anatomical axes, thus providing the ultrasound images with a correct frame of reference to aid interpretation of the images. While the calibration methods herein provide significant advantages, they are optional.

One of the advantages of methods and systems herein is that they can, by restricting the movement of the transducer about at least one axis or point, generate a 3D volume using feedback from an orientation sensor and without the use of a position sensor. Orientation sensors are widely available in a very small form factor and relatively inexpensive, while position sensors are relatively more expensive and add complexity to the system.

An additional advantage of some (but not all) of the methods and devices herein is that they can augment, or be used with, existing ultrasound systems that are capable of acquiring and displaying 2D image data (a majority of existing systems and probes only have 2D imaging capability, but some have a 3D mode as well). Once augmented, the ultrasound systems can then be used to generate 3D ultrasound image volumes of a subject, and viewed in real-time or near real-time, or those volumes can subsequently be visualized using a variety of 2D and 3D display methods. These embodiments provide a relatively simple and low-cost way of generating beneficial 3D volumes of a patient using existing 2D ultrasound systems. While not limited in use, these embodiments can be important in low-resource settings, including rural areas and the developing world. They may of course be used in developed regions as well, or in any setting or application where a more cost-effective solution is beneficial. As used herein, an existing ultrasound system generally refers to an ultrasound system that includes an ultrasound probe (with transducer therein), hardware and software for generating and/or processing ultrasound data, and a monitor for displaying ultrasound images. A majority of existing ultrasound systems and probes are only capable of acquiring, generating, and displaying 2D data and images, but some existing systems are capable of 3D imaging, even if they are typically not used clinically in that manner. Existing ultrasound systems can, of course, include additional components and provide additional functionality. It is important to note that the augmenting of existing ultrasound systems as described herein is merely an example of using the methods herein, and the disclosure is not so limited.

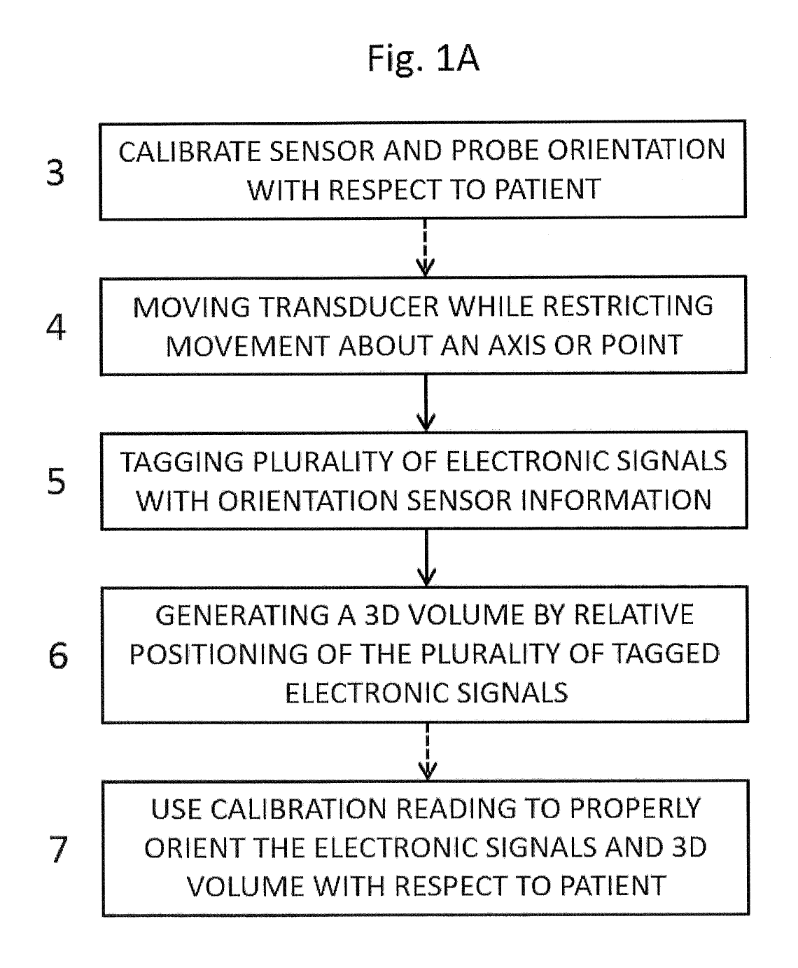

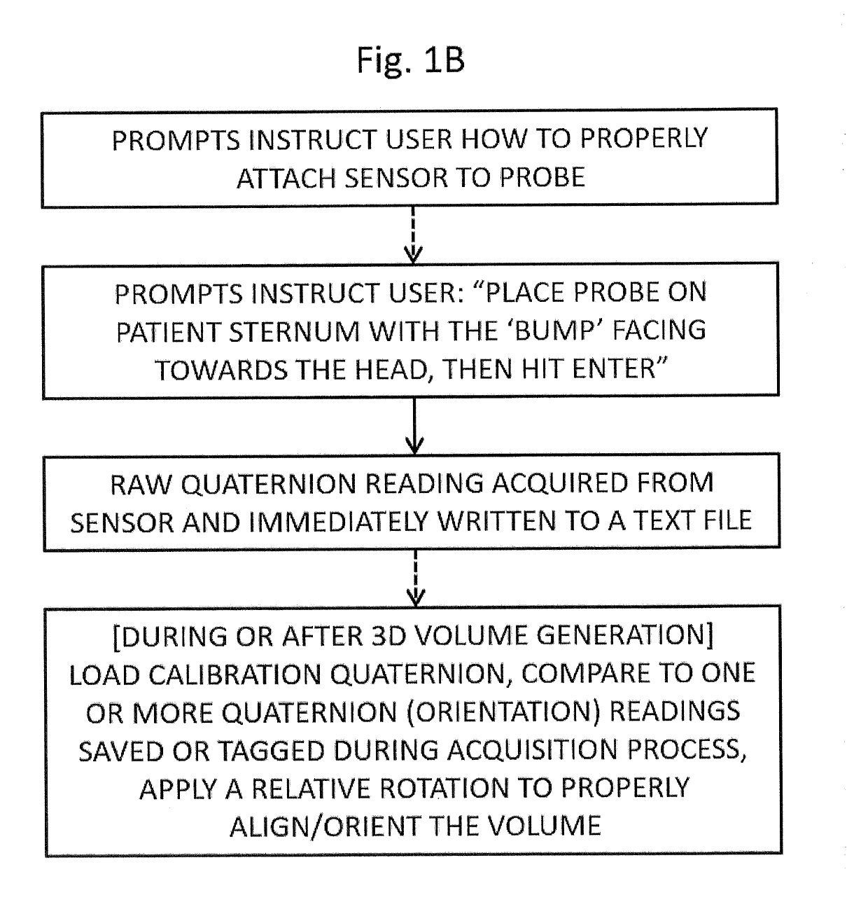

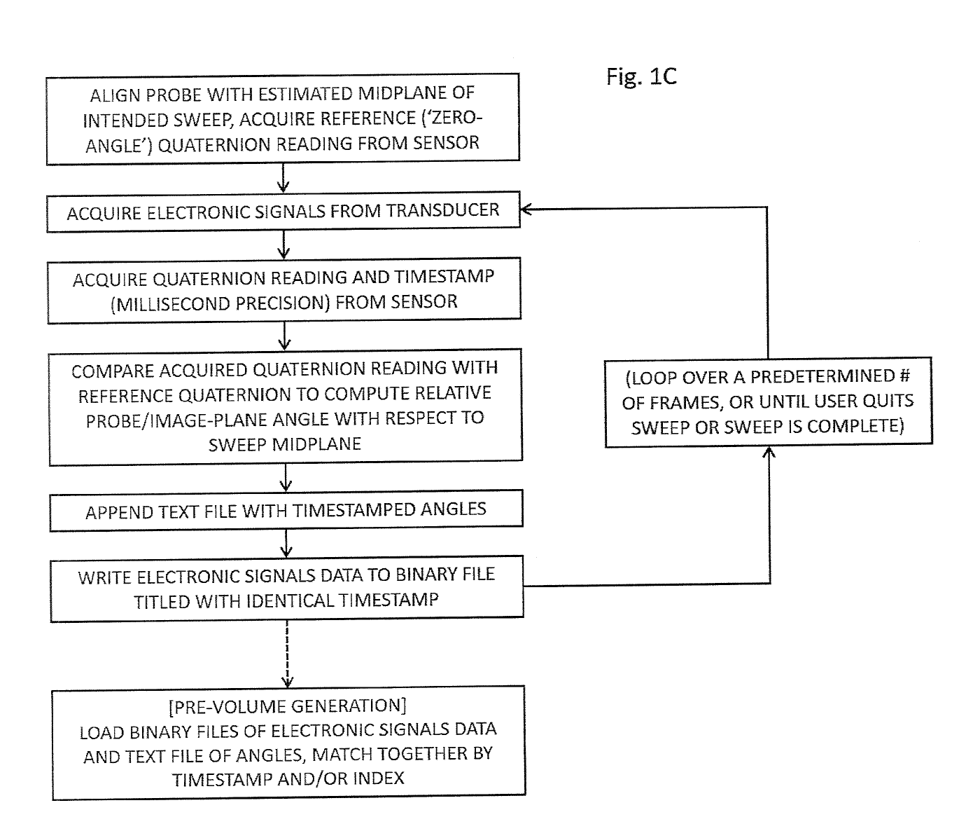

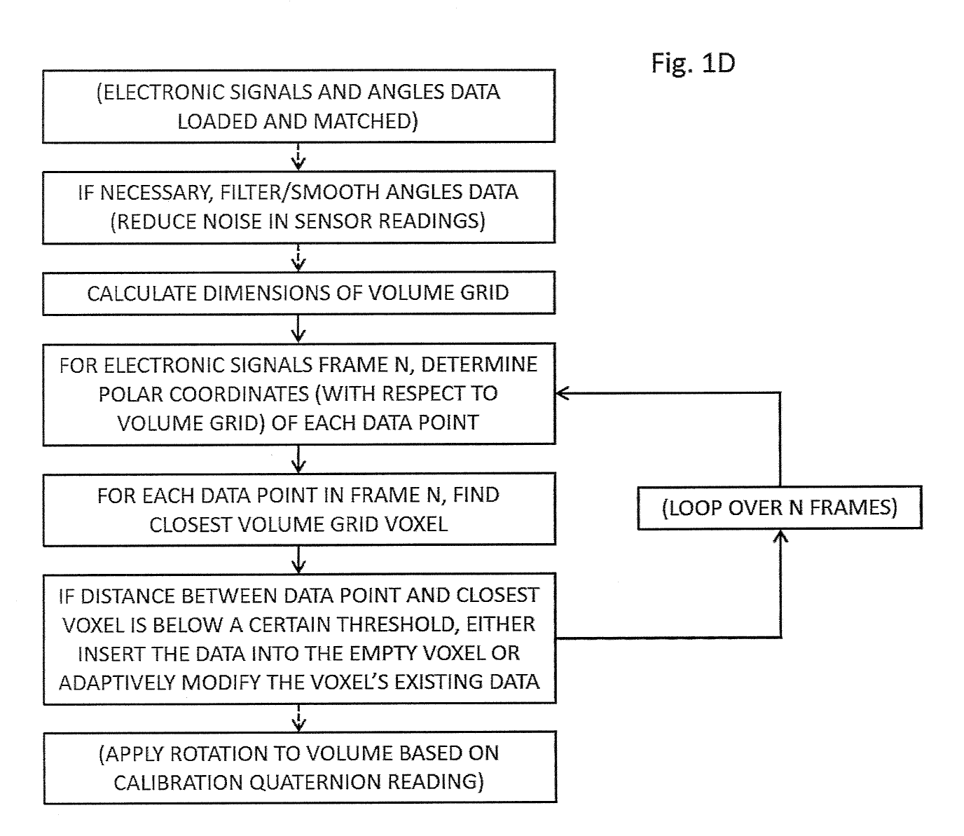

One aspect of the disclosure is a method of generating a 3D ultrasound volume, comprising moving an ultrasound transducer and an orientation sensor stabilized with respect to the ultrasound transducer, while restricting the movement of the ultrasound transducer about an axis or point, optionally due to an interface between an ultrasound probe and a movement restrictor; tagging each of a plurality of electronic signals indicative of information received by the ultrasound transducer, optionally 2D ultrasound image data, with information sensed by the orientation sensor, relative to the axis or point, each of the plurality of electronic signals indicative of information received by the ultrasound transducer representing a plane of information within the patient; and generating a 3D ultrasound volume image of the patient by positioning the plurality of tagged electronic signals indicative of information received by the ultrasound transducer at their respective orientations relative to the axis or point. FIG. 1A illustrates an exemplary method including steps 4-6, and optional calibration step 3 and optional using the calibration reading step 7.

The methods of use herein allow for freehand movement of the probe, meaning that a person can move the probe with her hand, about an axis or point.

FIG. 1B illustrates a exemplary calibration method, which is referenced herein but is described in more detail below. The calibration method in FIG. 1B is merely exemplary and does not limit the disclosure herein. Modifications to this exemplary calibration method can be made. For example, the method in FIG. 1B can be modified to exclude some steps or include other steps.

By restricting the movement of the transducer about a particular axis or point, each of the electronic signals indicative of information received by the ultrasound transducer can be tagged, or associated with, real-time information sensed by the orientation sensor (e.g., an angle) relative to the particular axis or point. The axis or point is thus a reference axis or point, and the electronic signals indicative of information received by the ultrasound transducer, tagged with orientation data, can then be used to generate a 3D volume relative to the reference axis or point. For example, the tagged electronic signals indicative of information received by the ultrasound transducer can be inserted into a 3D voxel grid along a plane at an appropriate angle relative to the axis or point.

FIG. 1C illustrates a merely exemplary tagging process performed while using the ultrasound probe (e.g., sweeping), not all steps of which are necessarily required. Other tagging methods can be used herein, and the merely exemplary method in FIG. 1C is simply to illustrate a tagging process, and the disclosure is not limited to the specific method in FIG. 1C or the particular steps in this exemplary method. As shown, the probe is aligned with an estimated midplane of the intended movement (i.e., “zero angle”), and a reference quaternion reading is obtained from the orientation sensor. Electronic signals are acquired from the transducer (which are described in more detail below) and a quaternion reading and timestamp are acquired from the orientation sensor (which occur simultaneously), and the quaternion reading and timestamp are tagged to the frame of electronic signals. The method compares the acquired quaternion reading with the reference quaternion reading to compute a relative probe/image-plane angle with respect to the midplane. In this particular embodiment a text file is then tagged with timestamped angles, and the electronic signals data is written to a binary file titled with the identical timestamp. The method loops over a predetermined number of frames, or until the user stops the sweep or until the sweep is complete. The exemplary tagging method can optionally, as part of a pre-3D volume generation step, load binary files of electronic signals data and text files of angles, and match them together by timestamp and/or index. FIG. 1C shows a particular, illustrative, tagging process, and not every tagging process herein includes every step. The order of the steps is not necessarily limited to those herein.

FIG. 1D illustrates a merely exemplary 3D volume generation method, utilizing the tagged data from the tagging method in FIG. 1C, or other suitable tagging process. Other 3D generation methods can of course be used, and the disclosure is not limited to this merely exemplary 3D volume generating method. The method optionally loads and matches electronic signals and angles data. Optionally, the angles data can be filtered/smoothed, which reduces noise in the orientation sensor readings. The method calculates dimensions of the volume grid. For an electronic signals frame, the method determines polar coordinates (with respect to the volume grid) of each data point. For each data point in the frame, the method finds the closest volume grid voxel. If the distance between the data point and the closest voxel is below a certain threshold, the method either inserts the data into the empty voxel, or adaptively modifies the voxel's existing data (e.g., averaging the data with the existing voxel data). The method loops over the number of frames and repeats those steps. Optionally, the method applies rotation to the volume based on the calibrated quaternion reading. Tagging and 3D generation methods are described in more detail below, and FIGS. 1C and 1D are meant to introduce the concepts in the context of the overall disclosure.

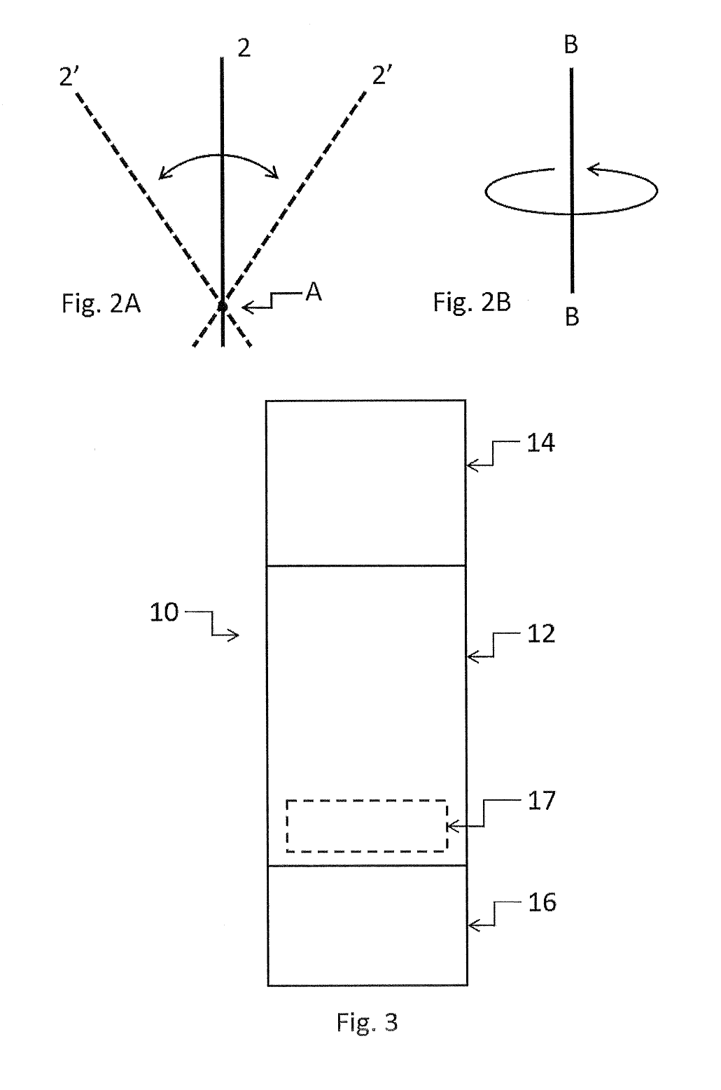

“Movement” about an axis or point, as used herein, includes any movement with respect to an axis or point, such as rotation, pivoting, tilting, spinning or twisting, and freely tumbling about a point. Freely tumbling refers to moving the transducer in multiple dimensions, methods of which generally require using all coordinates/dimensions of the orientation sensor's quaternion orientation reading. FIGS. 2A and 2B illustrate exemplary types of movement restriction, with FIG. 2A illustrating restricting object 2 to rotation about axis A (extending into and out of the page) to different positions 2′. FIG. 2B illustrates spinning or twisting an object (not shown) about axis B-B.

When movement is restricted as described herein, the movement can be restricted by any object that can restrict movement about a particular axis or point. For example, movement may be restricted by a mechanical fixture, or a hand (or fingers) of medical personnel or the patient. For example, medical personnel can “pinch” the sides of an ultrasound probe with two or more fingers, thus using fingers as a movement restrictor to restrict movement about an axis or point. In some embodiments the movement restrictor is the patient's body. For example, in transvaginal ultrasound applications, the patient's body can act as the movement restrictor. In some embodiments the movement restrictor is part of, or integral with, the ultrasound probe. That is, the movement restrictor can be any feature or mechanism built into the probe that allows for restricted movement about at least one particular axis or point.

One aspect of the disclosure is a 3D ultrasound image volume generating apparatus, comprising: an ultrasound probe in a fixed position relative to an orientation sensor; a movement restrictor configured so as to restrict the movement of the ultrasound probe about a particular axis or point; a tagging module adapted to tag each of a plurality of frames of electronic signals indicative of information received by the ultrasound transducer, optionally 2D ultrasound image data, with information sensed by the orientation sensor, relative to the particular axis or point; and a 3D volume generating module adapted to position each of the plurality of orientation tagged frames of electronic signals indicative of information received by the ultrasound transducer at respective orientations, relative to the particular axis or point, to generate a 3D volume image.

FIG. 3 illustrates an exemplary schematic of a merely exemplary apparatus 10 that includes an ultrasound probe 12 (with transducer 17 therein), an orientation sensor 14, and a movement restrictor 16. The orientation sensor 14 has a position that is fixed relative to ultrasound transducer 17 inside probe 12, and in embodiments herein the sensor has a position fixed in relation to both the transducer and probe. Movement restrictor 16 is, in this embodiment, configured to interface with probe 12 and is configured such that movement restrictor 16 restricts the movement of probe 12 about at least one axis or a point in response to a user (e.g., medical personnel) moving the probe. Movement restrictor 16 is also configured such that it can be positioned on the body of a patient.

FIG. 3 is a schematic and is merely an example of an apparatus, but this disclosure is not so limited. For example, the orientation sensor can be in any relative position to the transducer, and in some embodiments the orientation sensor is inside the body of the probe. Additionally, the movement restrictor can be integral with, or built into the body of the probe. The disclosure thus also includes an ultrasound probe that includes the orientation sensor therein, as well as can function as the movement restrictor to restrict movement of the transducer about at least one axis or point.

One of the advantages of systems and methods herein is that they can generate 3D volumes using information sensed by an orientation sensor, and do not require information sensed by a position sensor (i.e., an x, y, z sensor). In fact, in the embodiments herein, the systems and methods (unless indicated to the contrary) specifically exclude a position sensor (although information from a position sensor can conceivably be used with modification to the systems and methods). Examples of commercially available position sensors (which are not needed) include optical, electromagnetic and static discharge types. An electromagnetic version includes a transmitter (which may be placed on the transducer), and three receivers (placed at different, known locations in the room). From the phase shift difference in the electromagnetic signals received by these three receivers, the location and orientation of the ultrasound transducer can be determined. Such sensing methods require expensive equipment external to the sensing device for triangulation purposes, and these can cause electromagnetic interference with other medical equipment commonly found in hospitals and clinics. Additional disadvantages of some of these sensor types and their use include that the scanning room must have these sensors installed and the system calibrated, before actual scanning can occur, and that they have limited range—the receivers can only be used with accuracy with about 2-3 feet of the transmitter box.

Orientation sensors (which may also be referred to as an angle, or angular, sensors) are of a type that sense rotation about a single or multiple axes, including, but not limited to, capacitive MEMS devices, gyroscopes, magnetometers, sensors employing the Coriolis force, and accelerometers. The orientation sensors are capable of providing real-time feedback data corresponding to the probe's angular orientation. Any number of inertial modules (for example, one may employ a 3-axis gyroscope, a 3-axis magnetometer, and 3-axis accelerometer-components, which are common in many modern smartphones) are capable of this and are commercially available for relatively low cost. The orientation sensors may also be adapted to transmit sensed orientation information wirelessly. Orientation sensors are generally inexpensive compared to position sensors and their use, which is why the systems and methods herein, which can generate 3D volumes using only sensed orientation information and do not need sensed position information, provide a more cost-effective and simplified solution than other approaches to 3D ultrasound generating that include position sensors. Off-the-shelf orientation sensors can be used in the systems and method herein. Alternative embodiments that are modified relative to those herein could include a position sensor, but would not have advantages of systems and methods herein.

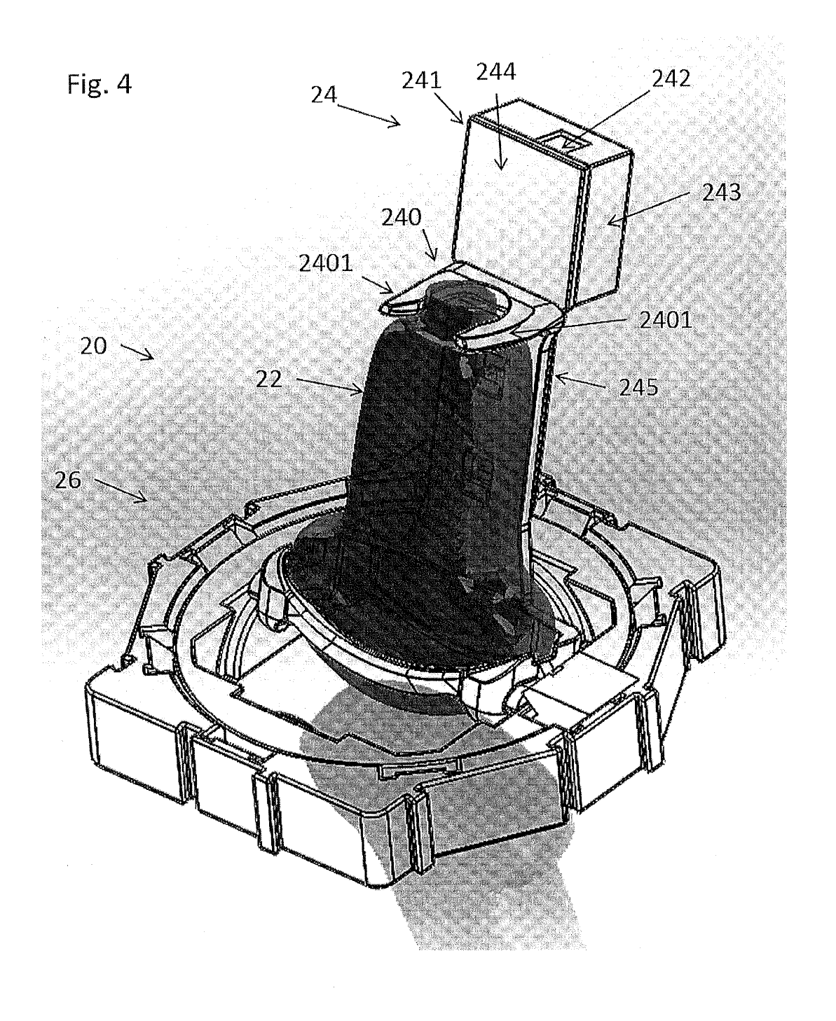

FIG. 4 illustrates a portion of a merely exemplary ultrasound 3D volume generation system. FIG. 4 illustrates apparatus 20, which includes movement restrictor 26, ultrasound probe model 22 (cable not shown for clarity), and sensing member 24. Movement restrictor 26 is described in more detail below. Sensing member 24 interfaces with probe 22 such that the position of an ultrasound transducer within probe 22 is fixed relative to an orientation sensor of sensing member 24. In this embodiment, sensing member 24 includes ultrasound probe interface 240, which is configured to be secured to probe 22, and in this embodiment is configured to be secured to a proximal portion of probe 22. Sensing member 24 also includes housing 241, which can be integral with (manufactured as part of the same component) ultrasound probe interface 240, or they can be two separate components secured together. In this embodiment probe interface 240 and housing 241 are generally orthogonal to one another, but in other embodiments they can be in a non-orthogonal relationship (and the methods can correct for the non-orthogonal relationship). Housing 241 includes orientation sensor 243 secured to backing 244. Extending from backing 244 is elongate member 245, which is this embodiment has at least one feature that interfaces with the cradle (described below) that allows the sensing member to be removably attached to the cradle. As is described in more detail below with respect to the exemplary cradles, the sensing member can thus be easily moved from a first cradle to a second cradle (or any other mechanical movement restrictor), depending on the probe that is being used. Housing 241 also includes a communication interface 242, and in this embodiment is a USB port.

Sensing member 24 is configured to be secured to probe 22 so that the position of orientation sensor 243 is fixed relative to the ultrasound transducer once sensing member 24 is secured to probe 22. In this embodiment probe interface 240 is configured so that it can be attached directly to a proximal region of probe 22 and stabilized to probe 22, but can be easily removed from probe 22 at the end of the procedure. In this embodiment probe interface 240 includes two stabilizing arms 2401, and probe interface 240 is a deformable material. The stabilizing arms are spaced from one another, and the interface 240 is deformable enough, such that as the interface 240 is slid onto the proximal region of probe 22, the stabilizing arms deform away from one another as they pass the largest diameter region of the proximal region of probe 22, but as they interface 240 continues to be advanced, the arms will again move towards one another and towards their as-manufactured spacing. Arms 2401 help secure the probe interface 240 of sensing member 24 to probe 22, and thus help secure sensing member 24 to probe 22. FIG. 4 illustrates a mere exemplary way to secure an orientation sensor to an ultrasound transducer, and other constructions can be implemented as well.

Any of the cradles herein, examples of which are described in more detail below, can include a probe interface 240 (or any other type of probe interface herein that fixes the position of orientation sensor and the transducer). That is, a sensing member can be integral with the cradle, or it can be a component separate from the cradle (whether it is stabilized with respect to the cradle or not).

One of the advantages of some of the sensing members herein is that they can be secured to many type of existing ultrasound probes, which allows the sensing member to be used at least near-universally with existing ultrasound systems. This can eliminate the need to redesign or reconfigure existing probes, or manufacture completely new or different probes, which can greatly reduce the cost of the methods of 3D volume generation set forth herein. Probe interface 240 is configured to be able to be secured with many different types of existing ultrasound probes, such as convex, linear, curvilinear, phased array, micro convex, T-type linear, biplanar, endolumenal (for example, endovascular), or endocavitary (for example, transesophageal, endovaginal or intrarectal), and have proximal regions (where the cord or cable begins) that are the same size or are similar in size. Arms 2401 are deformable so that they can be moved away from one another when securing sensing member 24 to probe 22, but have at-rest, or manufactured, spacing between them to secure the sensing member 24 to probe 22.

In some embodiments, the “diameter” of the opening in probe interface 240 is between 10 mm and 35 mm (such as between 15 mm and 30 mm), and may be sized in that manner to be able to accommodate many standard ultrasound probes. In some embodiments the probe interface is adjustable to allow it to be secured to a plurality of different sized probes. Some sensing members are, however, probe-specific, and as such can be sized and configured to be secured to specific types of probes. When diameter is used in the context of a probe interface opening, it does not require a circular opening; rather, diameter refers to the largest linear dimension across the opening. As can be seen in FIG. 4A, the opening in this embodiment has a general C-shape, and diameter refers to the largest linear dimension across the opening. In this embodiment interface 240 is snugly secured to probe 22. Probe interface 240 can be, for example, a deformable material such as a polymeric material, and can be molded with a particular configuration to be able to be secured to most standard ultrasound probes.

Securing sensing member 24 to the proximal region of the probes secures the sensing member to the probe, and it does not interfere with a user's movement of probe 22. This allows a user to be able to grasp the probe 22 body and use it as she normally would during a procedure, and still have the sensing member 24 secured stably thereto. The position of the sensing member 24 relative to probe 22, as well as its configuration, allows for near universal use with existing ultrasound probes. Medical personnel thus need not be retrained using new probes, and new probes need not be manufactured.

Sensing member 24 includes probe interface 240 and housing 241, which includes the orientation sensor(s). In other embodiments the sensing member can have different configurations or constructions, as long as an orientation sensor is included therein or thereon. For example, the probe interface could still have stabilizing arms, but those arms could have magnetic elements at their respective ends, to help maintain their “grasp” on the probe 22 when in use. Alternatively, the sensing member can be secured to probe with other securing mechanisms, such as, for example, one or more straps wrapped around one or more portions of the probe body, a temporary or permanent adhesive, or hook-and-loop closures. The type of securing mechanism can vary greatly, and can be any suitable mechanism, as long as the sensor's position is fixed relative to the transducer so that their relative positions do not change during data acquisition.

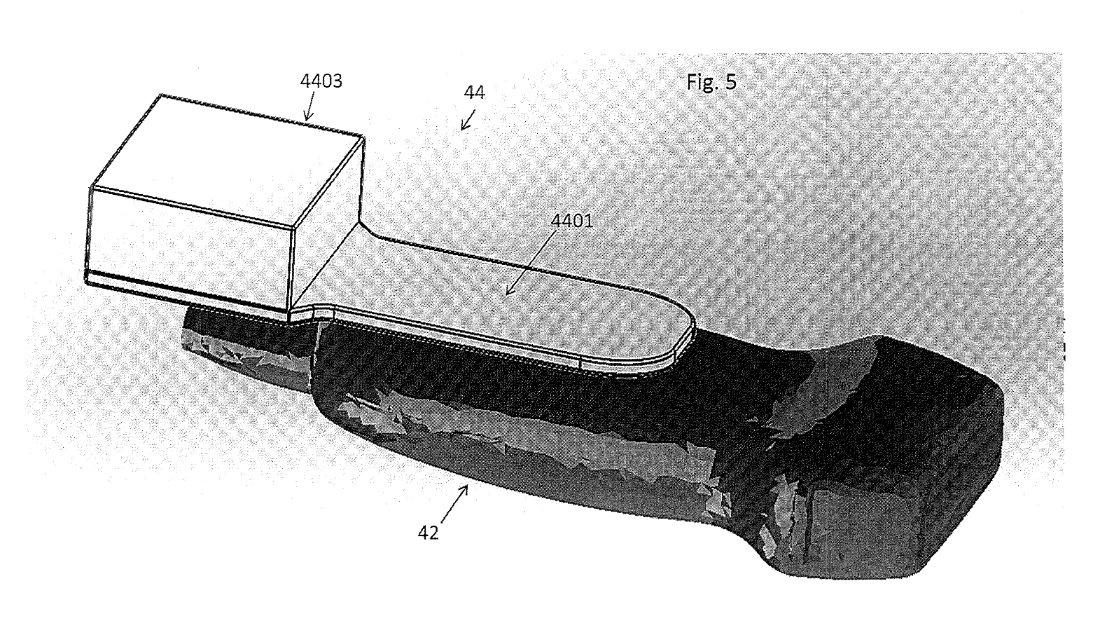

FIG. 5 illustrates another merely exemplary embodiment of an ultrasound probe secured to a sensing member. In this embodiment ultrasound probe (model) 42 is secured to sensing member 44. Sensing member 44 includes an orientation sensor 4403 secured to elongate member 4401, wherein elongate member 4401 can be secured to probe 42 by any number of, for example, straps (not shown), such as one being secured to a proximal region of probe 42, and one secured to a distal region of the handle portion of probe 42. FIG. 5 thus illustrates an alternative design of an ultrasound probe secured to an orientation sensor, where the position of orientation sensor is fixed relative to the ultrasound transducer within the probe.

FIGS. 4 and 5 are merely examples of ways to fix the relative positions of the transducer and sensor (if the sensor is not part of the probe), and the disclosure is not so limited. For example, some systems can include a sensing member that is adapted to be removably adhered to an ultrasound probe, or other component that can have a fixed position relative to the transducer. For example, in some embodiments the sensing member includes a relatively small circuit and wireless transmitter, wherein the sensing member wirelessly transmits the orientation information to a remote receiver (either in an existing ultrasound system or to a separate computing device in communication with an existing ultrasound housing). In use, a probe user could remove an adhesive backing, and adhere the sensing member to the ultrasound probe at any desirable position.

In FIGS. 4 and 5, the orientation sensor is secured to the ultrasound probe body, and is not disposed within the body of the ultrasound probe. In some alternative embodiments, the orientation sensor is embedded in the probe body. For example, an ultrasound probe can be manufactured with an orientation sensor within the body of the probe (with a fixed position relative to the transducer), and the orientation sensor can be in communication with an external device via the probe cable.

In some embodiments that modify the systems and methods herein, an orientation sensor may be optional, such as when orientation can be sensed from a component with known rotation (e.g., a motor). For example, the component that interfaces the ultrasound probe may also include motorized rotational stages to provide automated sweeps (for example, automated “twisting” or “fanning”). In this case, an orientation sensor may not explicitly be required to provide position, as an electronic motor may know exactly the amount of rotation being applied. The known amount of rotation can be used as part of the tagging procedure to tag each of the 2-D images.

As set forth above, methods herein include restricting the movement of the ultrasound probe about an axis or point while sensing orientation information relative to the axis or point. Restricting the probe's movement (whether it is rotating, twisting, tumbling, etc.) about a desired point or axis may be achieved in a variety of ways, and can be mechanical or non-mechanical (e.g., with fingers or a hand). Mechanical examples include, without limitation, features incorporated into the design of the probe housing itself, such as protrusions, indentations, rods, or wheels meant for holding or clamping the probe by hand or some other mechanism, a stand attachable to the probe that can provide a stable reference to the body surface, or by mating the probe with a fixture that can be positioned on the patient and interface with the probe.

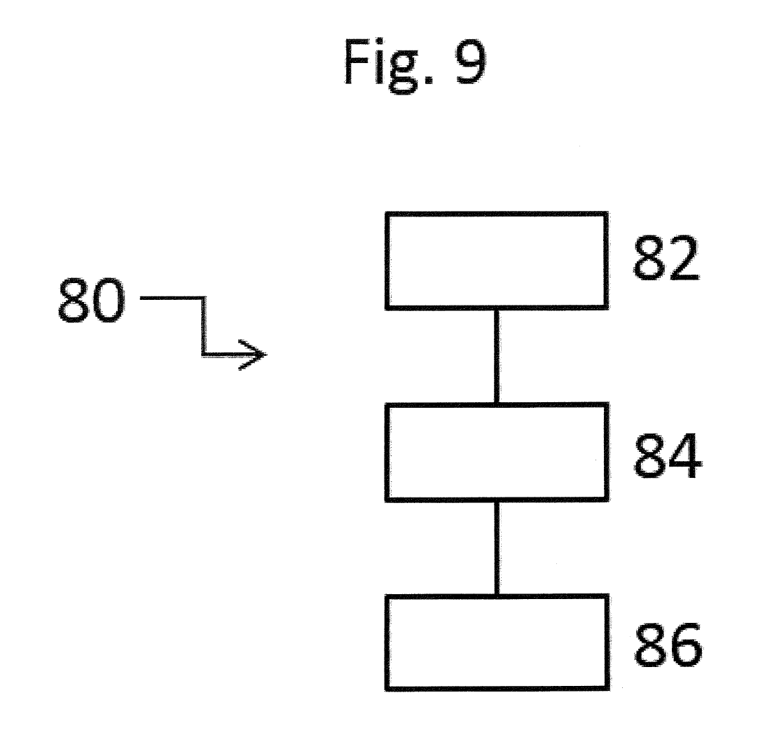

Such stands or fixtures may be adapted and/or configured to be positioned on and stabilized relative to the surface of the patient's body. For example, a fixture can be made of a material that is deformable to some extent, allowing for better conformation to the body. In other embodiments an adhesive (for example, using existing ECG adhesive stickers) can be used to provide additional stability between the fixture and the patient. In some embodiments the system can mechanically pull a local vacuum (creating suction), or have a bottom surface perforated with holes and a port to attach tubing from a vacuum line. These and other movement-limiting components and features can be made with relatively inexpensive materials (e.g., plastic), can be machined or manufactured using methods such as 3D printing (also see FIG. 9).

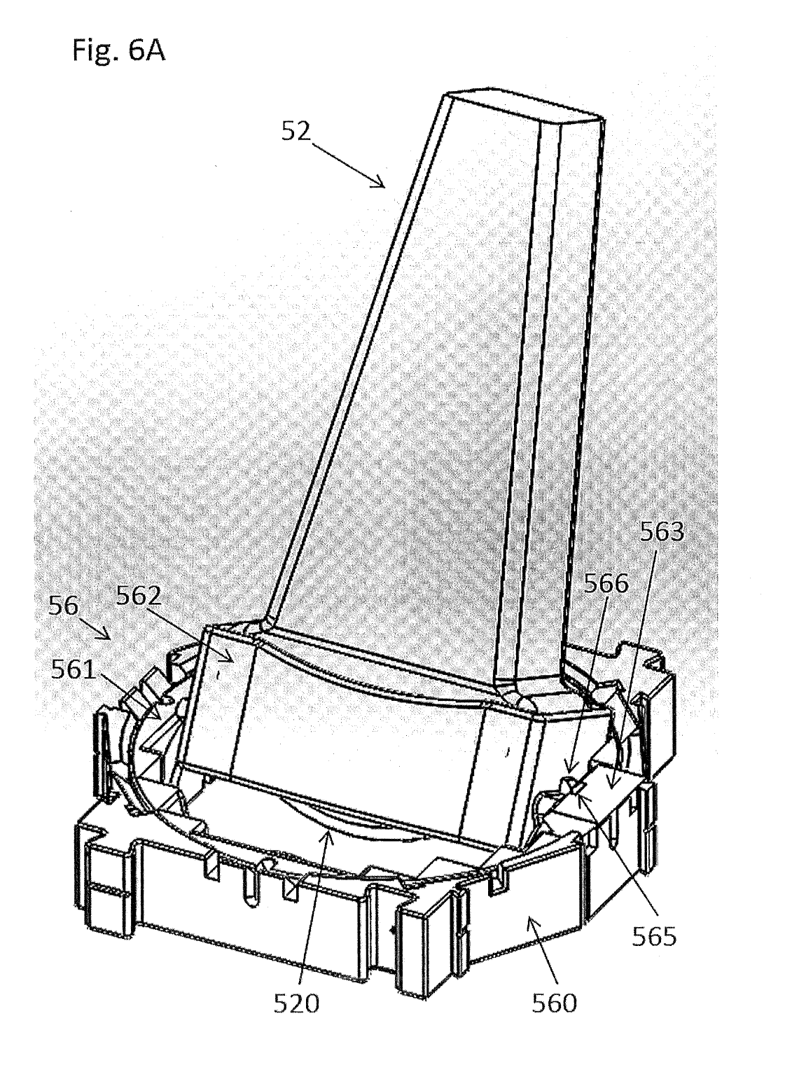

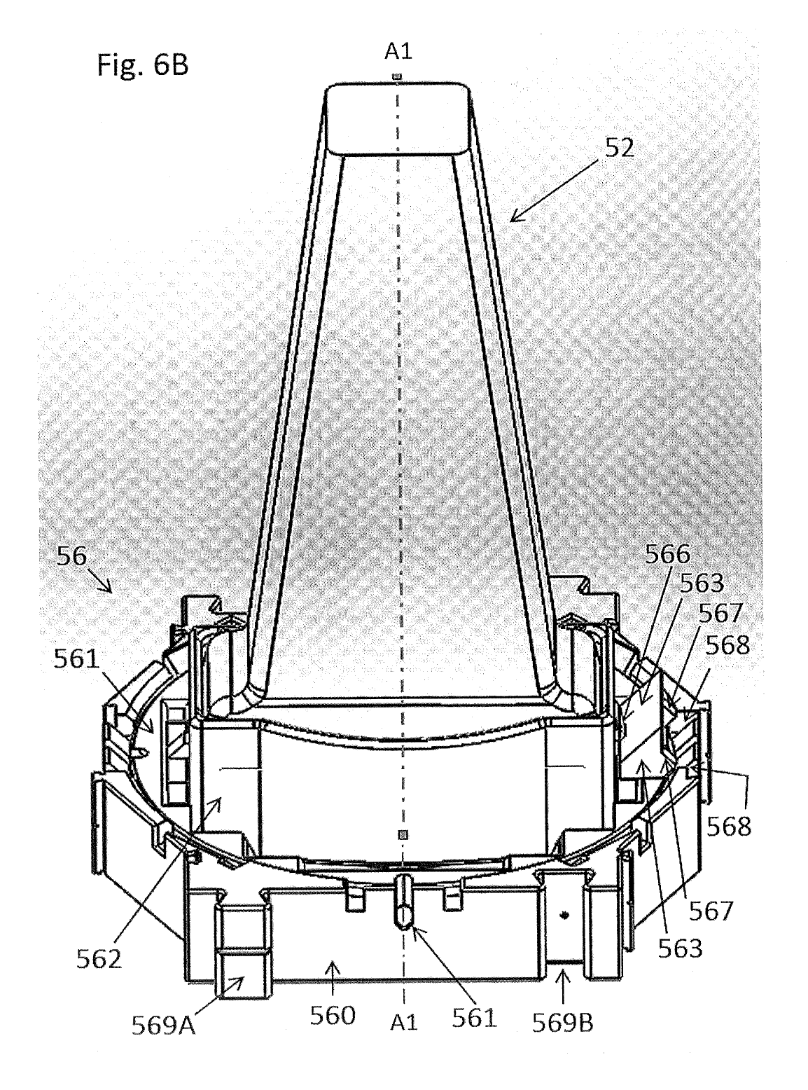

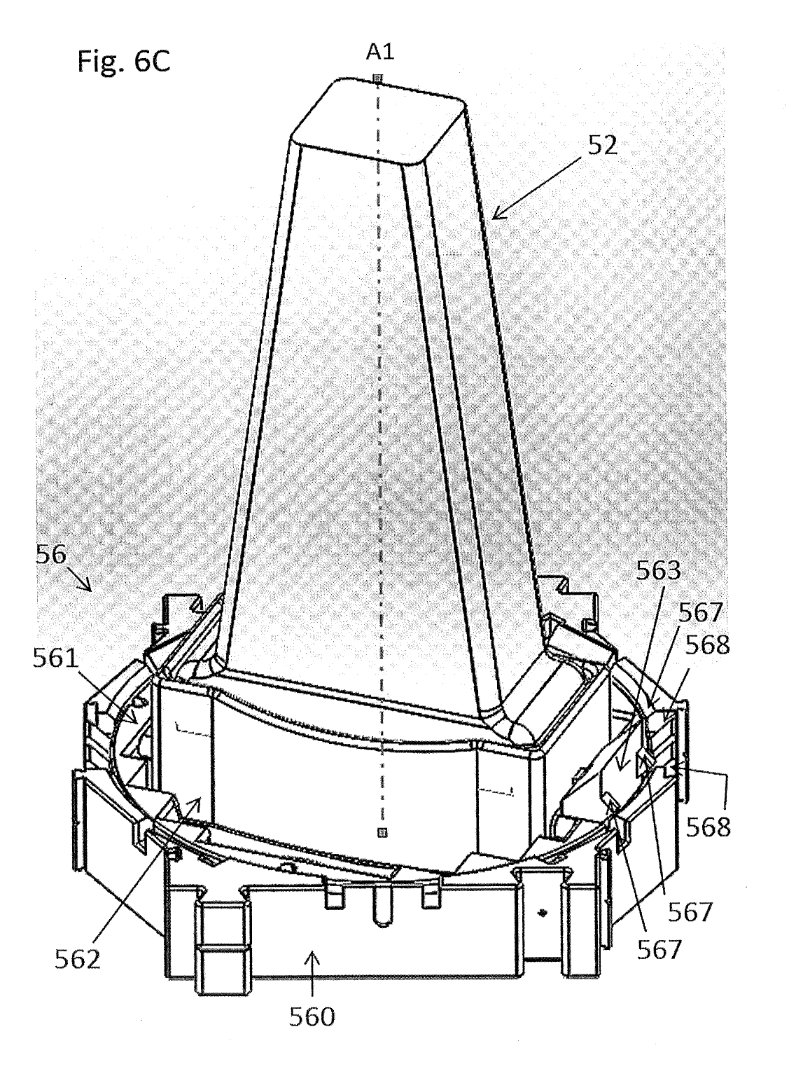

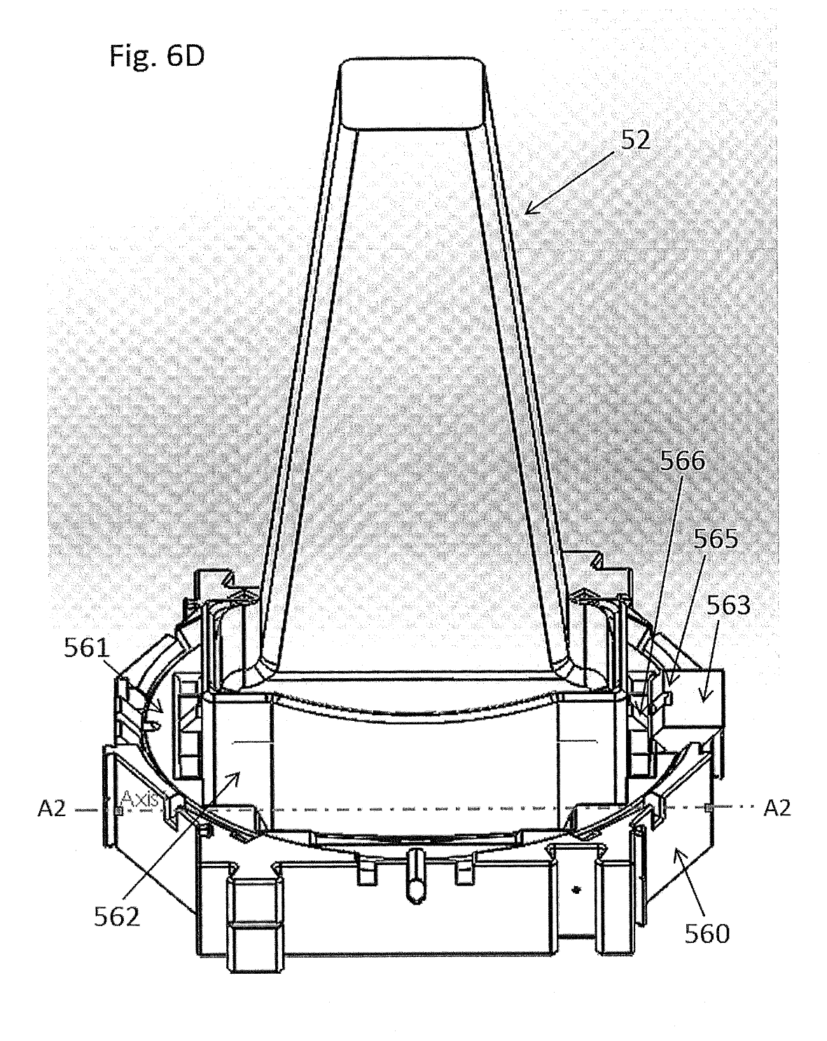

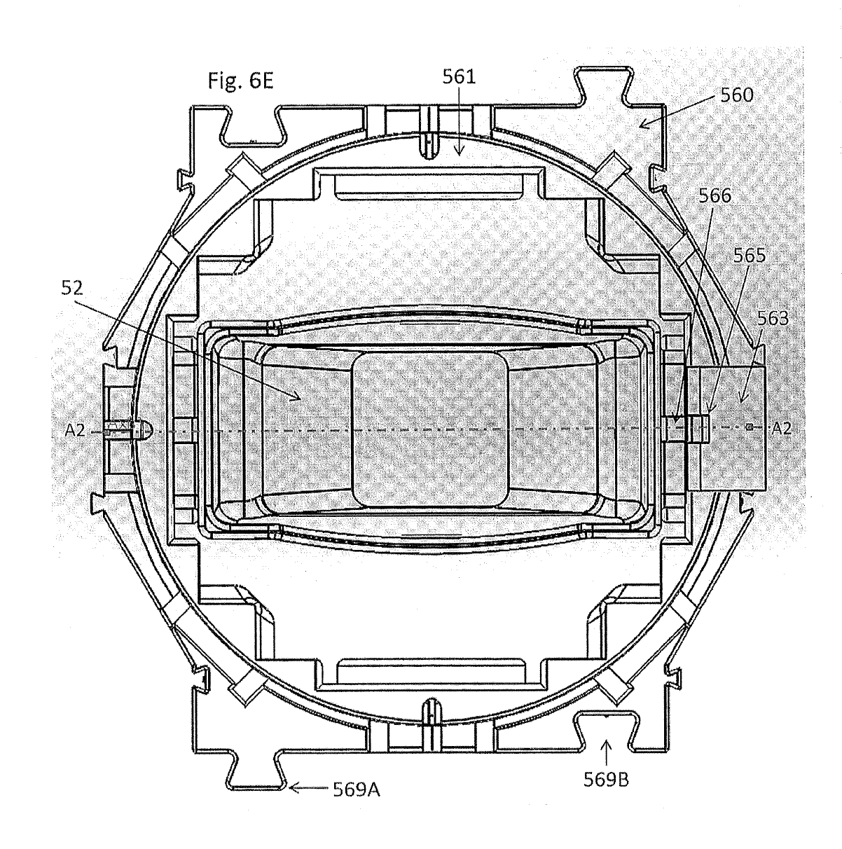

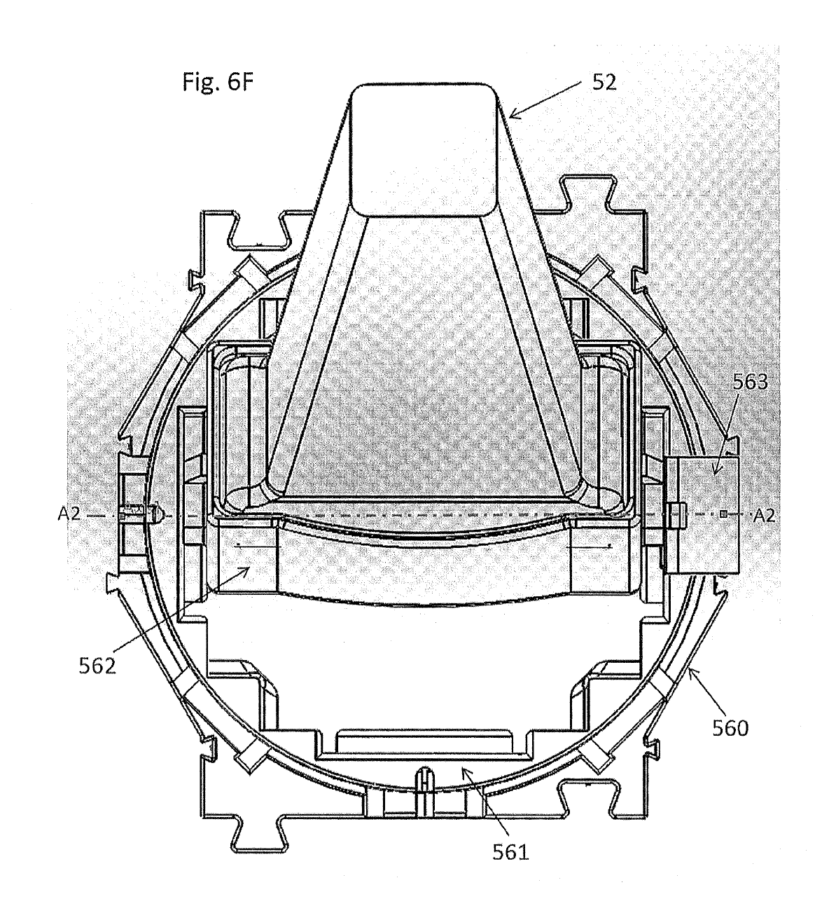

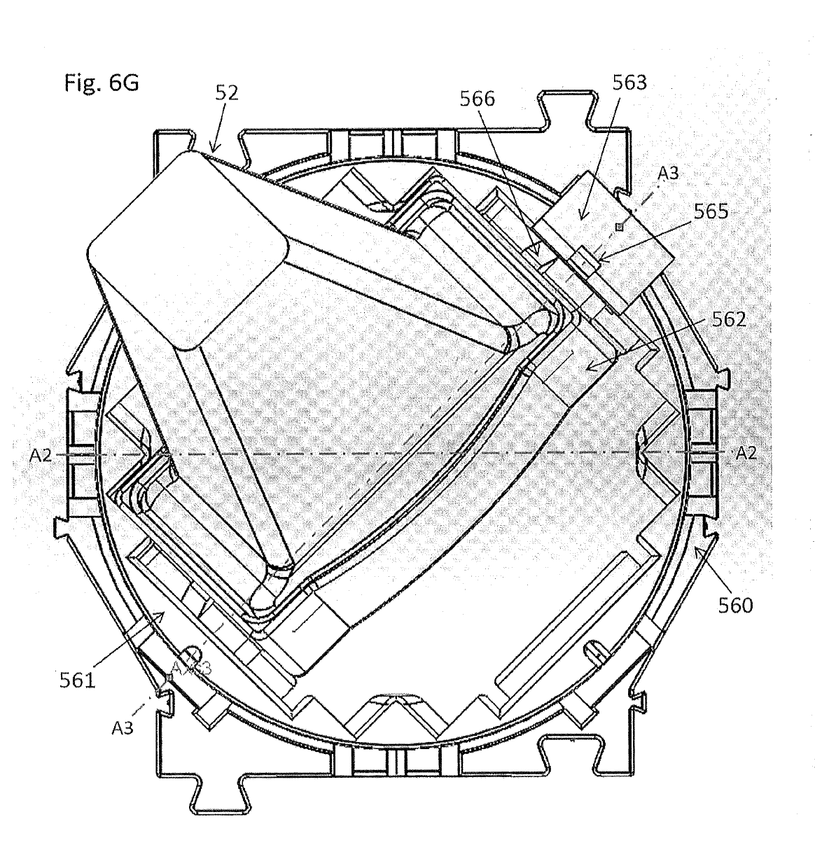

FIGS. 6A-6G illustrate a merely exemplary embodiment of a movement restrictor that is configured to interface with an ultrasound probe and restrict the probe's movement about one or more different axes or points (orientation sensor not shown for clarity). In some alternative embodiments, the movement restrictor can be, for example, part of the probe body. Movement restrictor 56 has a first state or configuration that restricts movement of a sensor-enabled ultrasound probe 52 about axis A1-A1 (see FIGS. 6B-6C), which is generally perpendicular to the body on which movement restrictor 56 is placed. Movement restrictor 56 is configured such that it can be modified from the first state or configuration to a second state or configuration that causes it to restrict the probe's movement about second axis A2-A2 (see FIGS. 6D-6F, which is generally horizontal, or generally parallel to the surface of the body. Movement restrictor 56 is also shown in FIG. 4. The movement restrictors herein can be adapted and configured to restrict movement about any number of axes or points, such as one, two, three, four, or more.

Movement restrictor 56 includes base 560, and slip ring 561, which is disposed within base 560. Movement restrictor 56 also includes probe cradle 562, which is configured to receive and stabilize ultrasound probe 52. Probe distal end 520 can be seen extending distally beyond probe cradle 562. Movement restrictor 56 also includes axis selector 563, which is adapted to be reconfigured relative to cradle 562 so that a particular probe restriction axis or point can be selected.

In FIGS. 6B and 6C, axis selector 563 is in a first locked configuration or state (in this embodiment in an “up” configuration) with probe cradle 562, in which axis selector 563 locking element 565 is in a locked relationship with cradle locking element 566 (FIG. 6A shows the locking elements 565 and 566 more clearly, but they are in an unlocked relationship in FIG. 6A). When the axis selector has thus been flipped “up” by a user (or automatically via a different mechanism), the locking interface between locking elements 565 and 566 stabilizes the probe (via its interface within cradle 562) in a generally upright, or vertical position. Slip ring 561 is adapted, however, to rotate within base 560 when axis selector is in the configuration in FIGS. 6B and 6C. Slip ring can rotate in FIGS. 6B and 6C because the two axis selector locking elements 567 are not engaged with base locking elements 568, as shown in FIGS. 6B and 6C. In this configuration, a user can thus spin, or rotate probe 52 only about axis A1-A1. Probe 52, probe cradle 562, slip ring 561, and axis selector 563 all rotate together. FIG. 6C shows the probe rotated relative to its position shown in FIG. 6B.

Movement restrictor 56 is also adapted to restrict the movement of probe about a second axis, A2-A2, when axis selector 563 is moved to a second state or configuration (different than the first state) relative to base 560. FIGS. 6D-6E show a second state, and in which axis selector has been moved “down” such that axis selector locking elements 567 are interfacing base locking elements 568 in a locked configuration. FIG. 6E is a top view. FIG. 6F illustrates the probe rotated relative to FIG. 6E. Axis selector 563 is also fixed in position relative to slip ring 561, and thus in this configuration axis selector 563 fixes the rotational position of slip ring 561 relative to base 560. Slip ring 561 can thus not rotate relative to base 560. In this configuration (shown in FIGS. 6A and 6D-6E, however, probe cradle 562 is free to pivot upon internal features of slip ring 561. Probe 52, stabilized within cradle 562, can thus be rotated by a user only about second axis A2-A2, shown in FIGS. 6D-6E. Movement restrictor 56 is thus a movement restrictor adapted to be positioned on a patient and adapted to allow a user to restrict movement about more than one axis or point. Movement restrictor 56 is also adapted to restrict the ultrasound probe's movement about one of the two axes, based on the user's selection.

FIG. 6G illustrates a third axis A3-A3 about which the movement of probe 52 can be restricted. Axis A3-A3 is 45 offset 45 degrees relative to axis A2-A2, as shown in FIG. 6G. FIG. 6G shows the slip ring 561, and thus the cradle and probe, rotated 45 degrees relative to FIGS. 6E and 6F. The base 560 is adapted to interface with the axis selector 563 to lock the axis selector in the position relative to the base (just as in FIGS. 6E and 6F). In this exemplary embodiment base includes locking elements disposed around the ring 561 at 0 degrees, 45 degrees, 90 degrees, 135 degrees, and 180 degrees, 225 degrees, 270 degrees, and 315 degrees. The axis selector 563 can thus be fixed relative to the base at any of those locations, thus fixing the probe movement about the corresponding axis. FIGS. 6D and 6E show probe restricted about axis A2-A2 (0 degrees), and FIG. 6G shows probe restricted about axis A3-A3 (45 degrees). While not shown, the probe's movement can also be restricted about the axis at 90 degrees, 135 degrees, of 180 degrees, which would require the slip ring to be rotated to the relative positions relative to the base. The other angles (225, 270, and 315 degree) could also be used, but they would be redundant to other angles. In this exemplar embodiment the movement restrictor can restrict the movement of the probe about five unique axes. In other embodiment the probe's movement can be restricted about any number of desired axes.

Movement restrictor 56, and other movement restrictors herein, may also be configured to restrict movement within a single image plane of the transducer, which could be helpful in, for example, scenarios in which it may be advantage to widen the field of view in-plane, such as in cardiac applications. Some cardiac probes have a relatively narrow aperture, and rocking back and forth in-plane could widen the field of view.

The movement restrictors herein can be configured to limit the movement about more than two axes (or in some cases only one axis).

In some alternative embodiments, however, a mechanical movement restrictor is not required to restrict the movement of the probe about a particular axis. For example, in some methods of use, a user such as medical personnel (or a second person assisting in the procedure, or even the patient) may be able to effectively pinch the sides of the probe with fingers, or another tool that is not interfacing the patient's body, creating enough friction to cause the probe to, when the probe is moved, only rotate about the axis defined by the axis between the fingers. The fingers in these embodiments are thus the movement restrictor. The disclosure herein thus includes restricting movement about a particular axis without necessarily using a mechanical movement restrictor. There may be advantages to using a mechanical movement restrictor, however, such as that the movement restrictor may be adapted to restrict movement about at least a first axis and a second axes.

In some embodiments herein the orientation sensor is secured to a component other than the probe, but is secured to have a fixed position relative to the transducer through the movement. For example, in some embodiments the orientation sensor is secured to a cradle, which in the embodiment in FIGS. 6A-6G, moves with the probe.

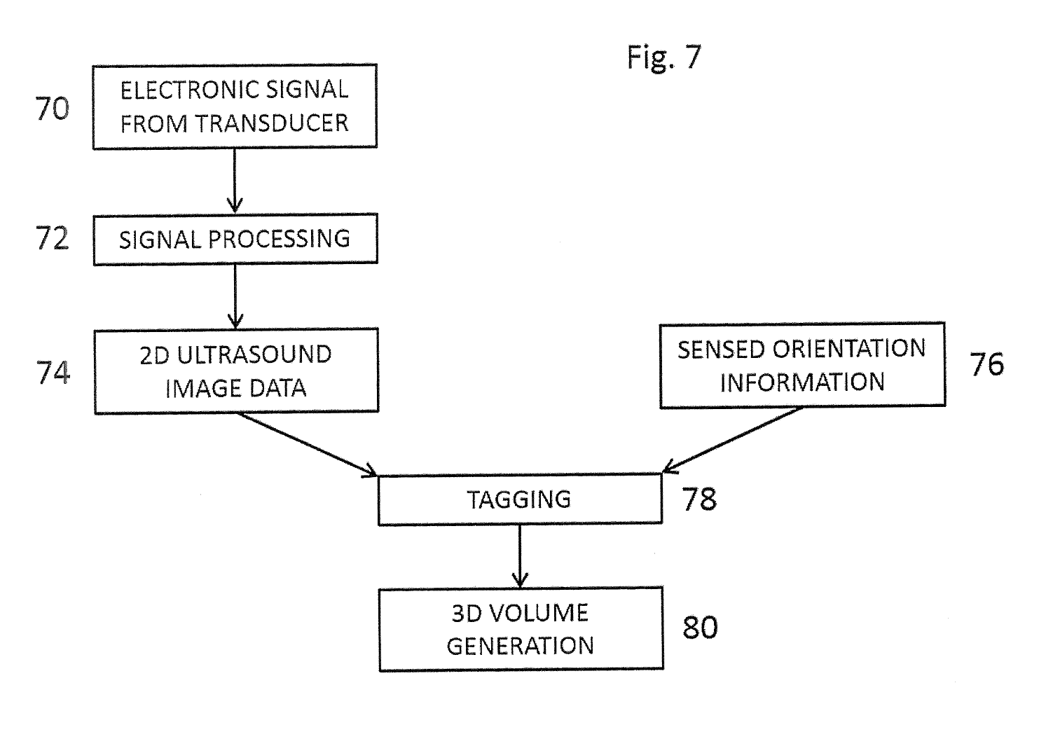

FIG. 7 illustrates a high level representation of data and information flow through exemplary methods, such as the methods shown in FIGS. 1A and 1C. Electronic signals received from the ultrasound probe (step 70) are generally referred to herein as raw channel data, and include radiofrequency (“RF”) data and in-phase (“I”) and quadrature (“Q”) data. I and Q data may be referred to herein as “I/Q” data. Signal processing at step 72 can include beamforming, envelope detection, and optionally scan conversion. Beamforming creates raw beamformed data, which can be RF or I/Q data. Envelope detection creates “detected” data, which may also be referred to herein as “pixel” data, and may be in any number of forms or formats, such as detected brightness-mode (B-mode) data, or scan-converted pixel brightness and/or color values. Outputs to signal processing step 72 thus include raw beamformed data (RF or I/Q) and detected data, which are included in the general term “2D ultrasound image data” as that phrase is used herein. Unless this specification indicates to the contrary, specific examples that describe “2D ultrasound image data” are referring to detected/pixel data. Any electronic data or information obtained at steps 70, 72 and 74 is referred to herein generally as electronic signals indicative of information received by the ultrasound probe. That is, raw channel data (RF or I/Q), raw beamformed data (RF or I/Q), and detected data are all examples of electronic signals indicative of information received by the ultrasound probe. A single acquired set of electronic signals indicative of information received by the ultrasound probe is referred to herein as a “frame” of data, regardless of the form of the signal, or the degree to which it has been processed (e.g., filtered, beamformed, detected, and/or scan-converted). For example, methods and systems herein can tag frames of raw channel, beamformed, and detected data.

Additionally, a “frame” of data can also be a 3D volume of data. For example, methods herein can be used with a matrix-array or wobbler probe and a 3D-capable scanner. In these embodiments the 3D frames of data (i.e., 3D volumes) that are internal to the scanner are tagged with orientation sensor information, using any of the methods and systems herein. In these embodiments, first and second (or more) 3D volumes can be used, based on the known orientation relative to at least axis or point, to generate, for example, a larger 3D ultrasound volume image. The concepts herein related to tagging frames of data can thus apply to both 2D data and well as 3D data.

When the phrase “electronic signals indicative of information received by the ultrasound probe” is used herein, it is describing a frame of data, even if the term “frame” is not specifically used.

In this particular embodiment, the tagging step 78 tags each of the plurality of 2D ultrasound image data with orientation information sensed by the orientation sensor (step 76), such as, without limitation an angle relative to the particular axis or point (additional exemplary aspects of which are shown in FIG. 1C).

A 3D volume is then, either in real-time or near-real time, or a later time, generated by software, step 80, that positions the plurality of tagged 2D ultrasound image data at their respective orientation relative to the particular axis or point. Exemplary details of a 3D generation method are also shown in FIG. 1D. The software that generates the 3D image volume also positions the plurality of tagged 2D ultrasound image data at their calculated positions within 3D space based on sensed orientation data and without the use of sensed position data.

In alternative methods to that shown in FIG. 7, the tagging step comprises tagging raw channel data received from the transducer (step 70 in FIG. 7), such as raw channel RF data or I/Q data, rather than 2D ultrasound image data.

The tagging and 3D generation methods can be performed with software that is added to existing ultrasound systems. That is, the methods can be incorporated with existing ultrasound systems, or added the manufacture of new ultrasound systems.

Alternatively, existing 2D ultrasound systems can be augmented with devices or methods herein to provide high quality 3D volumes, which greatly reduces the cost and avoids the need to update existing ultrasound systems or manufacture an entirely new ultrasound system. Existing 2D ultrasound systems already include an ultrasound probe and are already adapted to generate 2D image data (and display 2D images) based on echo signals received by the transducer.

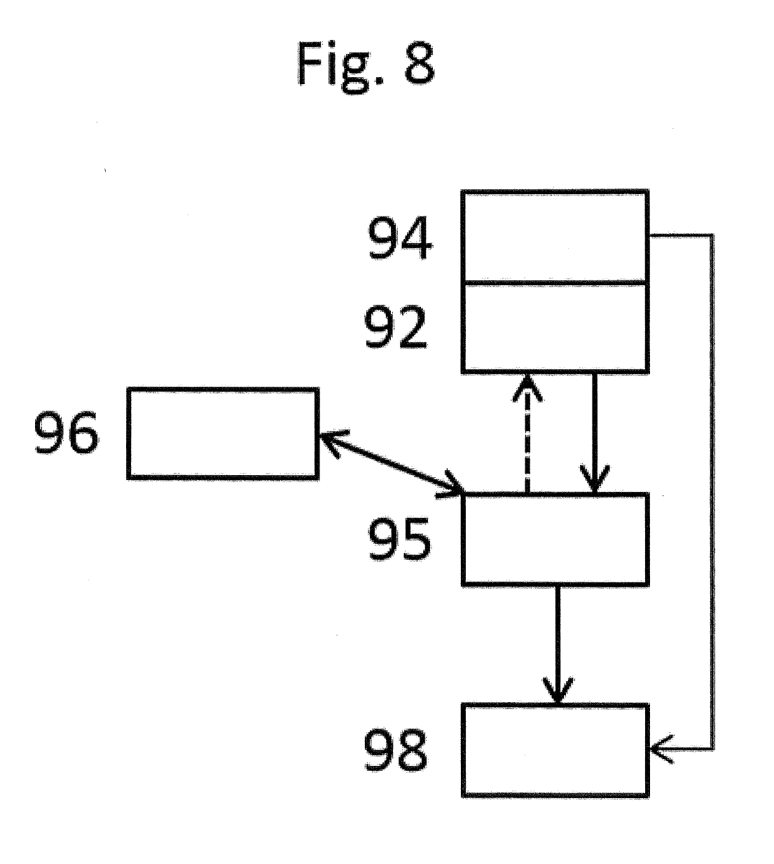

FIG. 8 illustrates an augmentation of an existing ultrasound system with an orientation sensor and an additional external device, which is adapted to generate the 3D volumes. The existing system includes ultrasound housing 95, probe 92, and display 96. Ultrasound probe 92 is shown secured to sensing member 94, which includes an orientation sensor. An external device 98 (e.g., laptop, tablet, or other similar computing device) is in communication (wired or wireless) with ultrasound system housing 95 and sensing member 94. In this embodiment, orientation information sensed from the orientation sensor 94 is received as input to external device 98, as shown FIG. 8. 2D ultrasound image data (e.g., detected data or raw beamformed data) can be taken from an external port or some other data port on the ultrasound system 95 and input to external device 98 (such as via an accessory cable or wireless adapter), which is shown in FIG. 8. External device 98 includes thereon software for tagging the electronic signals indicative of information received by the ultrasound probe, optionally 2D ultrasound image data, with sensed orientation data and for generating the 3D volume. External device 98 can have a display for displaying and interacting with the 3D volume. The external device 98 display can also function as a user interface to guide and/or facilitate user acquisition of data (e.g., prompts, instructions, configuration selections, modes, etc.) External device 98 can also have memory to store data or information, which can be used for any of post-acquisition 3D image volume generation (processing and reconstruction), visualization, and analysis.

Any of the information or data obtained at any step in the process can be stored in one or more memory locations for future use, including further visualization. Additionally, electronic signals indicative of information received by the ultrasound probe and sensed orientation data can be stored separately or together, and the 3D volume generation software can be adapted to generate the 3D volumes later based on the stored data.

Again, the exemplary system in FIG. 8 enables use of any existing ultrasound system capable of acquiring 2D image data, which reduces cost of the 3D volume generation. In this exemplary embodiment, the additional components that enable the 3D volume generation include the external device with tagging and 3D volume generating software, and a sensing member secured to the ultrasound probe.

The sensed orientation sensor information can be communicated from the orientation sensor to the external device in a wired or wireless manner. For example, in the embodiment in FIG. 4, the sensing member includes a USB or other communication port, which can be used to connect the sensing member and the external device. The sensed data can thus be communicated from the sensing member to the external device. The sensing member can alternatively be adapted for wireless communication with the external device, and communicate the sensed orientation sensor data to the external device wirelessly.

In alternative embodiments, however, it may be desirable to redesign existing ultrasound systems and probes to incorporate aspects of the systems and methods herein. An exemplary method of doing that is to include an orientation sensor inside a probe (rather than being a separate component secured to it), and the computing device of the ultrasound systems can be modified to include the tagging software and/or the 3D volume generating software (a separate external device is thus not a required aspect of this disclosure). The computing device on the ultrasound system would then receive as input the feedback from the orientation sensor (via the probe cable), and the tagging software and the 3D reconstruction method—using both the sensor feedback and the electronic signals indicative of information received by the ultrasound transducer (e.g., raw channel data, raw beamformed data, and detected data) already existing in the ultrasound system—can be disposed in the ultrasound system. The existing monitor can then display the 3D-generated volume, and the system can include updated user interface software to allow the user to interact with the visualization of the 3D volume as set forth herein. The user interface can be adapted to toggle the ultrasound monitor between 2D mode and 3D visualization modes.

FIG. 9 illustrates such an exemplary ultrasound system. FIG. 9 illustrates exemplary system 80 that includes a probe 82 (with transducer and orientation sensor therein), one or more housings 84 that comprises hardware for handling data (e.g., one or more of transmitter/receiver, beamformers, hardware processors, and scan converters) and software for signal processing and user interface, and display 86. In this and similar embodiments, the orientation sensor is disposed within the probe housing, and the tagging and 3D volume generation software are disposed within housing 84. Again, the tagging step can tag any of the frames of data indicative of the information received by the transducer, such as raw channel data, beamformed data or detected data.

Calibration

As set forth above (see FIGS. 1A and 1B), any of the methods herein can also include a calibration step that calibrates the orientation sensor (and thus the probe) with respect to the patient's anatomical axes (a frame of reference). The orientation sensor on, in, or near the probe can be used to take an orientation sensor reading to calibrate orientation relative to the patient and provide a frame of reference. FIG. 1B illustrates a merely exemplary calibration process. One optional step in the calibration process is to instruct the user how to properly attach the sensing member to the probe (if this step is applicable to the system being used). In an exemplary positioning step, when the patient is supine, the ultrasound probe (with the associated orientation sensor) face is positioned on the patient's sternum with the probe axis perpendicular to the patient's body, and an index marker (the “bump”) pointing toward the patient's head. The sensor reading can thus calibrate the orientation of the probe/sensor relative to one or more particular anatomic axes of the patient. Once the calibration reading is taken (see FIG. 1B), this information can be used to apply accurate labels of anatomical cardinal directions and/or planes to the live 2D images and/or the generated 3D volume with text (examples of which are shown in FIGS. 10B, 11B, 12B, and 13B) and/or a 3D graphic probe icon, which can tilt and spin in real-time to mimic the probe orientation with respect to the body (examples of which are shown in FIGS. 10A, 11A, 12A, and 13A), any and all of which may be saved with the 2D or 3D image (see FIG. 1B). The calibration reading can also be used to auto-flip or auto-rotate the live 2D image in response to changing probe orientation, to provide a consistent frame of reference for the user. The calibration reading can also be used so that warnings can be displayed to alert the user of, for example, an uncommon or unconventional probe orientation. The calibration reading can also be used to transform the coordinate system of the 3D volume to match that of the patient (the patient's cardinal anatomical axes), or alternatively be used to aid a re-sampling of the 3D volume to a voxel grid aligned with the patient's cardinal anatomical axes, so that the physician can then step or “pan” through a stack of slice images, which are transverse, sagittal, or coronal, in a fashion similar to reviewing 3D datasets from CT or MRI imaging.

The calibration step can be used with systems that are not adapted to or do not generate 3D volumes. The calibration step and the associated methods of use can be beneficially used with existing 2D image systems. For example, the calibrating step can be used to provide a visual indicator on the 2D image of how the probe is oriented with respect to the patient.

FIGS. 10A-13B illustrate an exemplary process and benefit of an optional but highly advantageous step of calibrating the sensor and probe orientation with respect to the patient. FIG. 10A illustrates an exemplary calibration position of the face of the probe 150 (with sensor attached) on the sternum 152 of the patient, with the index bump towards the head of the patient. Using the sensor's reading taken from this calibration position, FIG. 10B illustrates, in real or near-real time, the 2D volume image, annotated visually with a label 153 of the anatomical plane of the patient (Sagittal in this figure), anterior (“A”) direction label 154, posterior (“P”) direction label 155, head (“H”) direction label 156 (optionally “CE” for cephalad, or “CR” for cranial), and foot (“F”) direction label 157 (optionally “CA” for caudal). Anybody looking at the image in FIG. 10B thus knows immediately in which plane the image is being obtained (or was obtained, if the data is stored), and the relative positions of the head and feet of the patient, as well as the anterior and posterior directions of the patient. These methods can thus automatically embed orientation information into the image, vastly improving the utility of such images (whether 2D ultrasound images or 3D volume images).

While 10A does illustrate the calibration position of the probe, the illustration in FIG. 10A (as well as FIGS. 11A, 12A, and 13A) is actually an exemplary orientation graphic that can be displayed on the monitor (can be shown live or saved with the image) to illustrate the position of the probe relative to the patient, so that someone viewing the image will quickly understand how the probe was oriented relative to the patient when the data was captured that was used to generate the image also being displayed.

FIGS. 11A and 11B illustrate probe 150 moved relative to patient 152, such that the imaging plane label 163 indicates in real-time, transverse/sagittal oblique, right side/head label 166 (alternatively cranial or cephalad), left side/foot label 167 (alternatively caudal), anterior label 164, and posterior label 165.

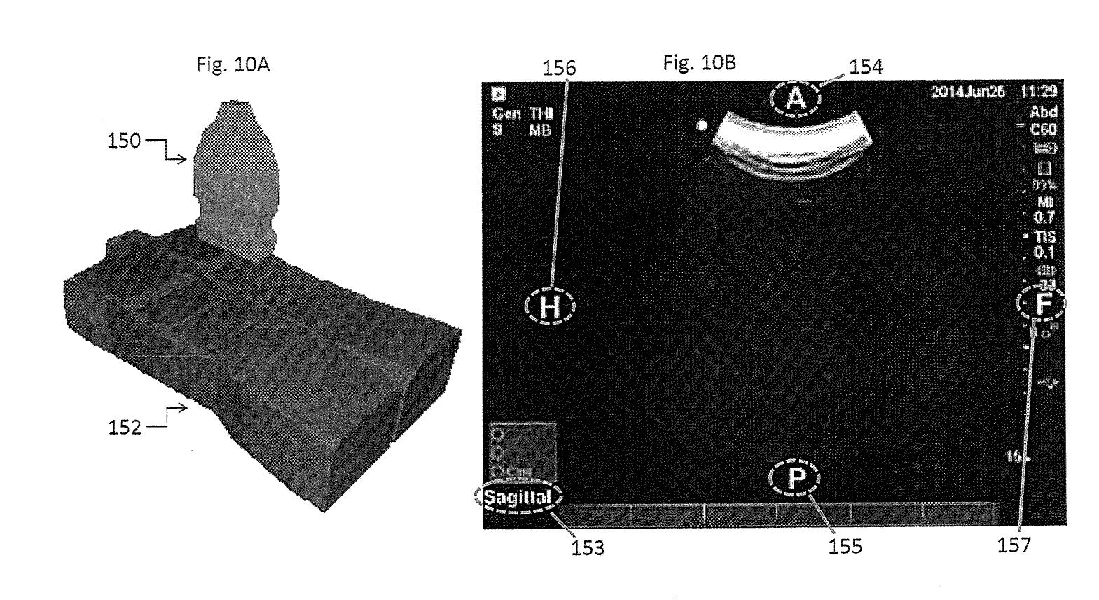

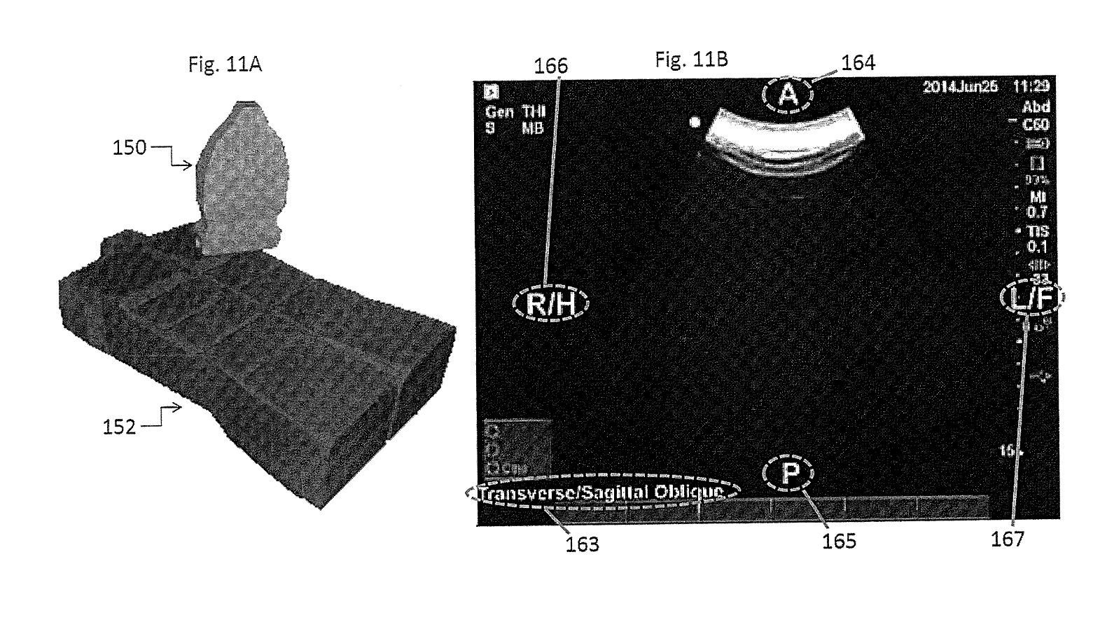

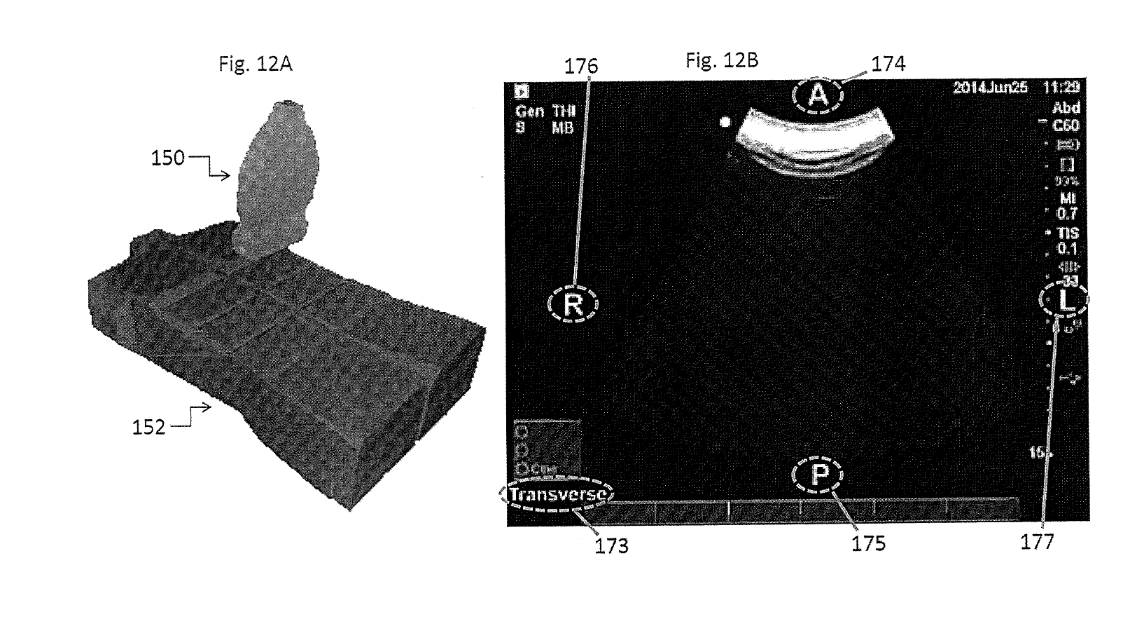

FIG. 12A shows probe 150 moved relative to patient 152 to be imaging in the traverse plane, and FIG. 12B shows the real-time 2D image, as well as plane label 173 (transverse), right label 176, left label 177, anterior label 174, and posterior label 175.

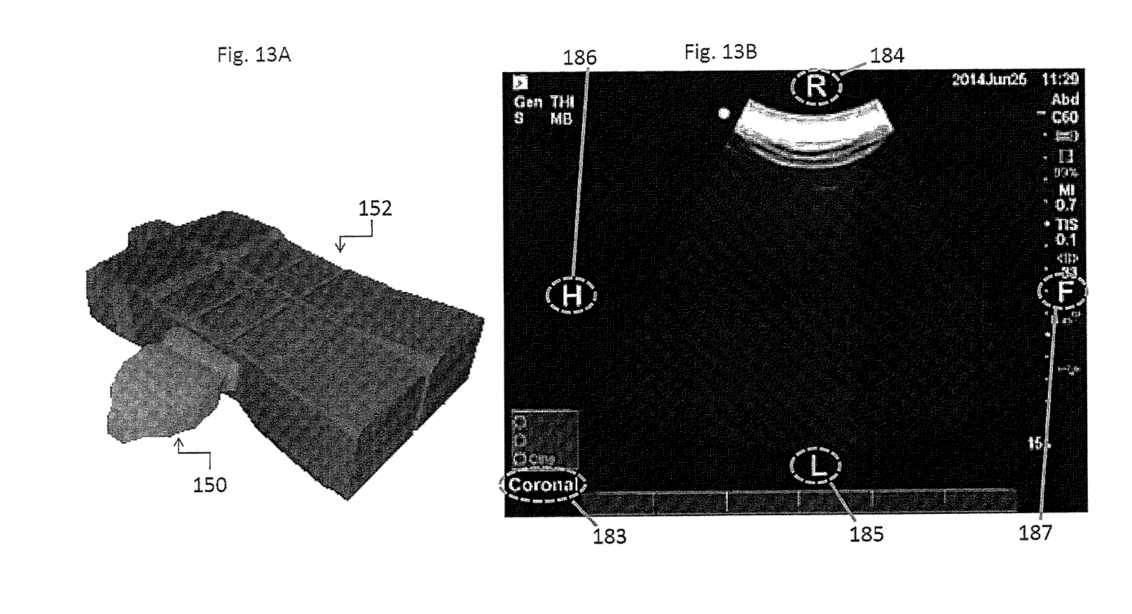

FIG. 13A illustrates probe 150 moved relative to patient 152 to be imaging in the coronal plane. FIG. 13B illustrates a real-time 2D image with anatomical plane label 183 (coronal), head label 186, feet label 187, right side label 184, and left side label 185.

FIGS. 10A-13B thus illustrate how valuable the optional calibration step and subsequent automatic image labeling with at least one of the anatomical plane and relative direction labels (e.g., any of head/foot, right/left, and anterior/posterior). 2D images and/or 3D volumes can be labeled in this manner, with at least one of the imaging plane and direction labels.

3D Volume Combining

An exemplary advantage of some of the methods and systems herein is that they allow for restricted movement about more than one axis (see, for example, FIGS. 6A-6G, and 10). In use, the same volume of tissue can be scanned/swept over by the ultrasound probe and image plane multiple times by rotating the probe about the different axes or points (such as an axis generally parallel to the body surface and an axis generally perpendicular to the body surface). The software can then use the plurality of 3D image volumes, or 3D volume data, and perform volume combining (e.g., compounding) techniques, which combine the image acquisitions from different ultrasound transmit and/or receive apertures to reduce speckle noise (i.e., the grainy background texture in ultrasound images), and improve image contrast and resolution. The disclosure herein thus includes software methods that can combine multiple 3D image volumes to increase the quality of the 3D volume, such as using coherent compounding (e.g., plane wave, synthetic aperture, etc.), and incoherent compounding. Image combining also enables the removal of image artifacts and barriers to sound transmission, which commonly and substantially limit visualization of structures with conventional 2D ultrasound. By combining image data acquired from multiple complementary acoustic windows into a single merged 3D volume, regions acoustically shadowed (for example, by bowel gas and/or bone) can be replaced or preferentially merged with valid image data. This can dramatically enhance the image quality and diagnostic information provided by the ultrasound images, potentially eliminating the need for CT or MR imaging.

First and second 3D volumes can be generated using ultrasound transducers that are operating at different frequencies. For example, high frequency ultrasound probes operate at relatively higher frequency, provide higher image resolution, and image at shallower depths. Lower frequency probes operate at lower frequencies, provide generally lower resolution, but have a better depth of penetration. The 3D volumes, generated using probes with different frequencies, can be compounded, taking advantage of the higher resolution at shallower depth, with the better depth of penetration of the lower frequency probe. In some embodiments the movement restrictor is configured to interface different types of probes with different frequencies, and is configured to restrict movement of each probe about at least one axis or point. For example, the system can include a restrictor with interchangeable cradles, each cradle configured to interface with a particular type of probe (or particular family of probes).

In some embodiments a user interface, on an external device on a modified existing ultrasound system, includes buttons (or similar actuators) or a touch screen that allow a user to select from the multiple axes. The user then performs the sweep about the axis or point, and the software saves that image data. The user can then select a different axis, and then performs the second sweep about a second axis or point. The software method can then compound the 3D image volumes, and the output is a higher quality 3D volume. Compounding in this context is generally known, and an exemplary reference that includes exemplary details is Trahey G E, Smith S W, Von Ramm T. Speckle Pattern Correlation with Lateral Aperture Translation: Experimental Results and Implications for Spatial Compounding. Ultrasonics, Ferroelectrics, and Frequency Control, IEEE Transactions on. 1986 May; 33(3):257-64.

Any of the methods herein can also include confidence mapping steps to assess 2D pixel quality prior to incorporating any of the 2D images into the 3D volume. Confidence mapping can also be used in any of the methods herein to preferentially select data from between at least two 3D volumes when combining/merging 3D volumes. Exemplary aspects of confidence mapping that can be used in these embodiments can be found in, for example, Karamalis A, Wein W, Klein T, Navab N. Ultrasound confidence maps using random walks. Medical image analysis. 2012 Aug. 31; 16(6):1101-12.

The disclosure herein also includes methods of use that merge, or stitch together, multiple 3D volumes (which may be adjacent or partially overlapping) to expand the total field of view inside the patient, thus generating a larger merged 3D image volume. This can enable the physician to perform a more complete scan of the body for immediate review, similar to CT but without the use of ionizing radiation. The plurality of 3D volumes can be merged, or stitched, together, as long as the relative position of each rotation axis or point is known or can be determined. In these embodiments, the 3D volumes can be partially overlapping, with a first 3D volume being at a different depth than a second 3D volume.

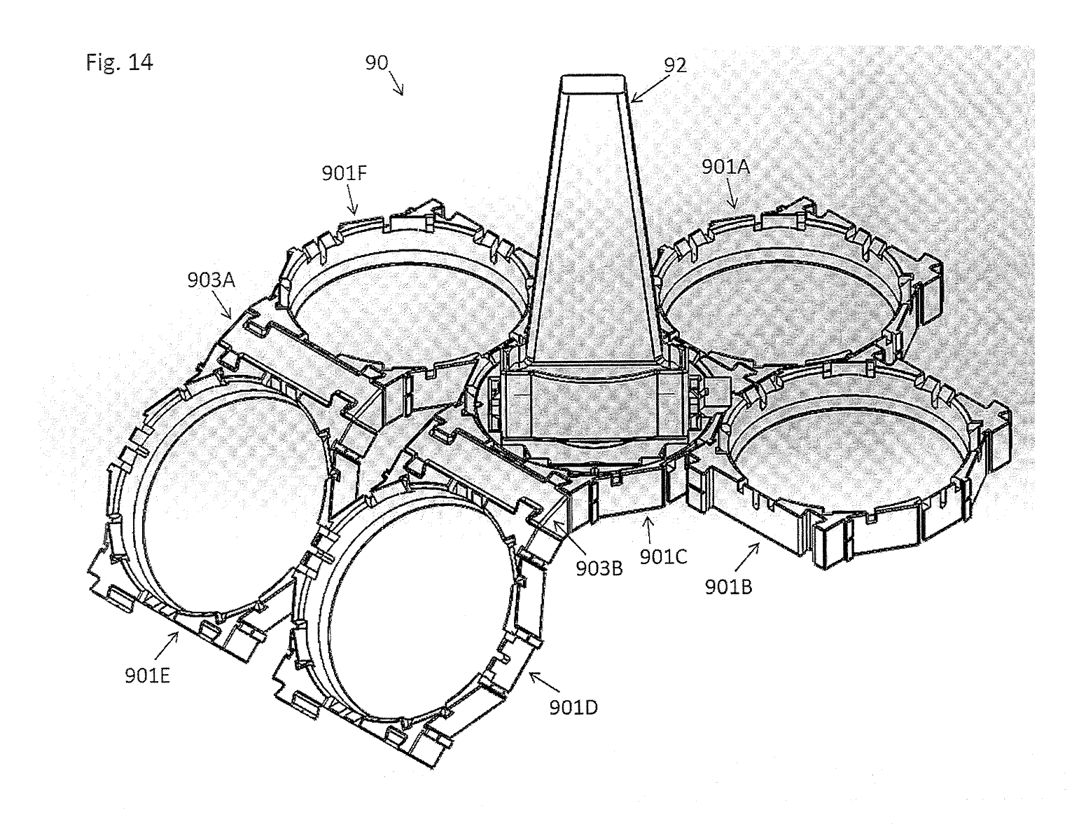

A merely exemplary apparatus that is adapted to enable multiple 3D volumes that can be combined (e.g, merged or stitched) together is shown in FIG. 14. FIG. 14 shows an apparatus that includes a plurality of movement restrictors 901A-F secured together. In this exemplary embodiment movement restrictors 901A-F are each the same as the movement restrictor in FIGS. 6A-6G. The description of FIGS. 6A-6G thus applies to this embodiment as well. Only movement restrictor 901C shows all of the components of the movement restrictors (e.g., base, slip ring, axis selector, probe cradle), while movement restrictors 901A, B, and D-F are illustrated only with the base component for clarity. Each base includes first and second linking elements 569A and 569B (see FIG. 6B) on a first side of the base, and third and fourth linking elements on a second side of the base, opposite the first side. In this exemplary embodiment the bases are hexagonal shaped, and two linking elements are on a first side of the hexagonal shape, while the third and fourth are on the opposite side. The linking elements allow for two bases to be secured together and stabilized with respect to the each other. In FIG. 14, the base of movement restrictor 901A is linked with the base of movement restrictor 901C due to linking elements 569A and 569B. The bases are also configured with additional linking elements that allow for movement restrictors to be linked in a close-packed configuration (e.g., the link between movement restrictors 901A and 901B, and between 901B and 901C). These close-packed linking relationships are enabled by linking elements on other sides of the hexagonally shaped bases. The bases are also configured with additional linking elements that allow for adjacent movement restrictors to be linked in a rectilinear configuration. Movement restrictors 901C and 901F are linked in a linear relationship. The bases are thus configured to enable a variety of configurations of the movement restrictors when linked.

The apparatus can also include one or more angled connectors 903A and 903B, which also have linking elements like the bases, and are thus adapted to interface with the bases. The angled nature of angled connectors allows adjacent movement restrictors to be coupled at an angle relative to one another (i.e., not aligned along a plane). This can be beneficial on a curved portion of the patient, where it is advantageous or necessary in order to engage the movement restrictor with the patient's body. The angled connectors can be used at any desired location to provide the relative angled coupling between adjacent movement restrictors.

Any number of movement restrictors may be linked together, in a variety of configurations, aligned or at an angle to one another, depending on the surface of the patient and/or the application.

Any of the bases can have configurations other than hexagonal, such as rectangular, square, circular, triangular, octagonal, or even irregular, such as if the shape or shapes are custom made for a particular use on the patient. The connectors can similarly have any suitable variety of configurations and linking members as desired.

In the embodiment shown in FIG. 14, each of the movement restrictors can have its own slip ring, axis selector, and probe cradle, or in some cases only one set is needed, and they can be removed as a unit and placed in other bases as the probe is moved with that particular movement restrictor.

In this embodiment, a probe 92 is shown stabilized in probe cradle associated with movement restrictor 90I C. The probe can be used in any of the manners described herein, such as moving the probe about one or both axes after selecting the particular axis with the axis selector. The probe has an associated orientation sensor (inside the probe or secured thereto), and the 2D images can be tagged as described herein (with orientation information and/or calibration information). After data has been obtained using one movement restrictor, the probe can be moved (and perhaps the entire slip ring/probe cradle, axis selector unit as well) to a different movement restrictor. The probe can be swept again about one or more axes or points. The probe can be moved to any number of movement restrictors to obtain image data. Information and data can be stored at any location at any or all steps in the process.

For a particular base, if sweeps about two axes are performed, image compounding can occur for each base before 3D volumes from adjacent movement restrictors are stitched.

Additionally, data can be saved after each sweep, and the software can process the data at any stage of the process.

In some embodiments the components interfacing the patient are fixed with respect to the patient. A user can simply hold the movement restrictors against the patient, or, for example, a temporary adhesive sticker or vacuum suction can be applied to hold the movement restrictors in place. In some embodiments, even if the patient interface component(s) is not specifically fixed with respect to the patient, software can correctly identify image landmarks to aid in stitching partially overlapping 3D volumes that were not acquired with the aid of a fixed mechanical reference system. Using a fixed mechanical system with a known configuration can, however, simplify and improve the accuracy of volume stitching.

In some embodiments the patient interface (e.g., the bases of the movement restrictors) can be a single integral unit. For example, in a modification of FIG. 14, one or more of the bases could be integral with one another (e.g., molded from a single mold), rather than discrete components that are linked together. For example, there may be certain shapes that work well for certain areas of the body regardless of the patient, and thus there may be an advantage of having a prefabricated base that allows for multiple probe positions.

The methods and devices herein (e.g., orientation sensor with restricted motion of the transducer) can also be used with synthetic aperture imaging. Synthetic aperture imaging requires RF data (channel or beamformed), which is obtained as described above. Synthetic aperture imaging can be performed by, e.g., saving 2D channel data for many different angular positions (e.g., using the apparatus in FIG. 14), and beam forming the ensemble of data on a point-by-point basis in 3D space. Using synthetic aperture imaging with the methods herein would advantageously generate high resolution images. The following references describe aspects of synthetic aperture imaging that can be incorporated into methods herein: Ylitalo, J. T. and Ermert, H. Ultrasound synthetic aperture imaging: Monostatic approach. IEEE Transactions on Ultrasonics, Ferroelectrics, and Frequency Control, 41(3):333-339, 1994; Frazier, C. H. and O'Brien, Jr., W. D. Synthetic aperture techniques with a virtual source element. IEEE Transactions on Ultrasonics, Ferroelectrics, and Frequency Control, 45(1):196-207, 1998; Karaman, M., Li, P.-C., and O'Donnell, M. Synthetic aperture imaging for small scale systems. IEEE Transactions on Ultrasonics, Ferroelectrics, and Frequency Control, 42(3):429-442, 1995. Some embodiments incorporate a combination of these methods incorporated by reference herein, but a preferred technique may be using the monostatic approach in the elevation dimension (rather than in the traditional scan plane). This could be coupled with the multistatic approach in the scan plane to generate extremely high resolution images/volumes.

Live Updating

Any of the methods herein can also be adapted to provide “live-updating” processing and/or display of the generated 3D volume with continued sweeping of the ultrasound probe. After a 3D volume has been generated (using any of the methods and systems herein) and the probe is still in use, the software is adapted to receive as input the current (i.e., live) 2D image data from the orientation-sensor-indicated plane and insert the current image data into the 3D data array, to add to, overwrite, or update the previous/existing data in the volume. The interface can optionally be adapted to display ‘past’ image data in the volume as dim or semi-transparent, and the current/live/most-recent plane of data can be shown as bright, highlighted, and/or opaque. The display thus allows the user to distinguish between the previous data and the current data. The live-updating volume display can provide guidance and confidence to users when performing intraoperative, invasive, or minimally-invasive procedures.

Additionally, some methods can provide near-live updating, rather than true live-updating. Near-live updating is intended to encompass all updates that are not true live updating. For example, near-live updating can replace the entire 3D volume during the procedure, or portions of the 3D volume, as new data is acquired.

Additional

The base 560 shown in FIGS. 6A-6G include guide 561, which in this embodiment is a needle guide. The guide can guide other devices such as a guide wire, catheter, etc. The guide can serve to allow a needle to be advanced into the patient while visualizing inside the patient, with either 2D images or 3D volumes. An exemplary beneficial use is that the needle can be inserted while visualizing the real-time images of the patient (either short-axis or long-axis) for more confident and consistent device placement, as well as improving the speed and safety of the procedure.

As set forth herein, the methods, devices, and systems herein enable much easier and more intuitive uses of ultrasound for many applications. Additionally, because of the speed, safety, portability, and low-cost of ultrasound relative to other imaging modalities (for example, CT or MRI), the 3D image volumes can be acquired quickly, and optionally immediately reviewed at the bedside post-acquisition, saved for later use or post-acquisition reconstruction, or sent electronically to a remote location for review and interpretation. Systems, devices, and methods herein also enables effective use and enhancement of existing low-end equipment, which is important in low-resource settings, including rural areas and the developing world, as well as cost-conscious developed world settings.

Whether real-time or not, interface and image functions such as thresholding, cropping, and segmentation can be performed to isolate and visualize particular structures of interest.

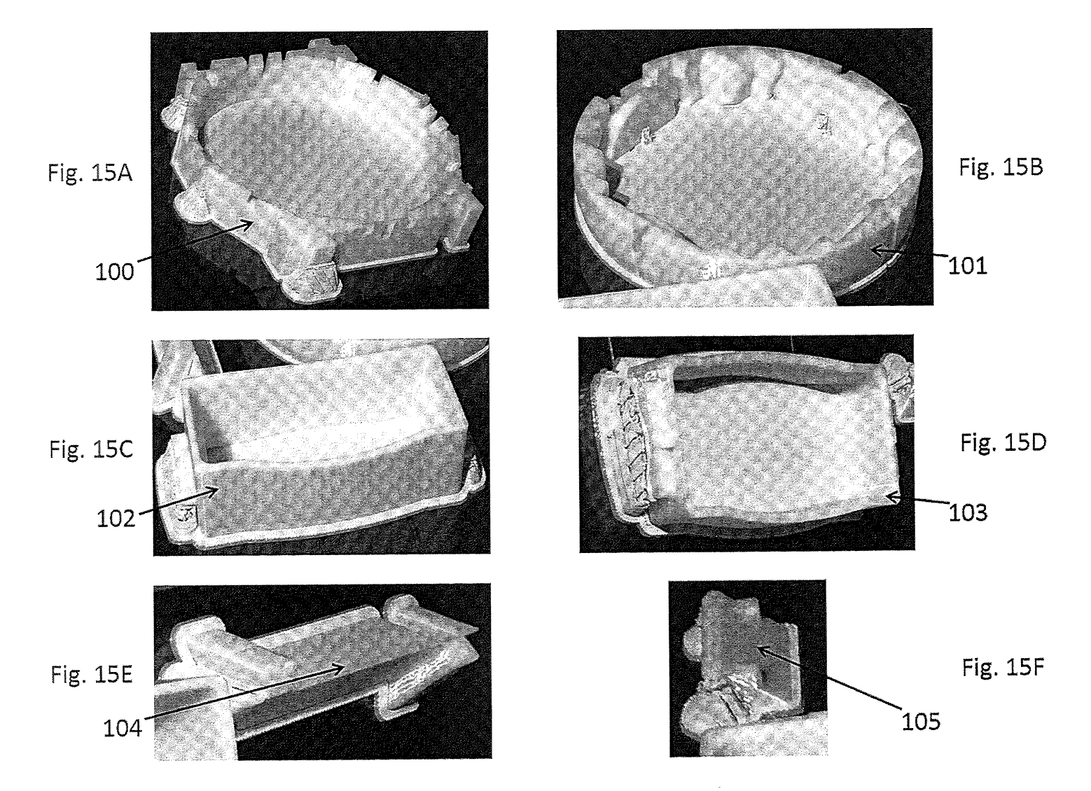

FIG. 15 illustrates components of the exemplary embodiments herein. FIG. 15 shows base 100 in FIGS. 6A-6G, slip ring 101 shown in FIGS. 6A-6G; cradles 102 and 103 (either one of which can be used in the embodiment in FIGS. 6A-6G); angle connector 104 from FIG. 14; axis selector 105 from FIGS. 6A-6G. The materials for these components can be selected to be somewhat deformable yet stiff enough to be able to maintain their shapes while any forces from probe movement are applied thereto. The components can be made using any suitable manufacturing technique, such as molding (e.g., injection molding or poured material molding) or 3D printing. If molds are used, the molds can be 3D printed.

The bottom surfaces (the surfaces that contact the body) of the any of the bases herein need not be flat, but can be molded with curvature to conform to certain body surfaces, if desired.

“Transducer” and ultrasound “probe” may be used interchangeably herein. Generally, an ultrasound probe includes an ultrasound transducer therein. When this disclosure references a “probe,” it is generally also referencing the transducer therein, and when this disclosure references an ultrasound “transducer,” it is also generally referencing the probe in which the transducer is disposed.

While the embodiments above describe systems and methods that rely on the ultrasound transducer within the probe as the energy source (i.e., sound pulses), the systems and methods herein are not so limited. This disclosure includes any method or system in which the energy source is not the ultrasound transducer. In these embodiments the ultrasound transducer can still function as a detector, or receiver, of acoustic data that occurs as a result of energy emitted into the tissue, whatever the source. Photoacoustic imaging is an example of such an application. Photoacoustic imaging involves exciting tissue with a pulsed laser. Scattering dominates light propagation, so unlike ultrasound, the light excitation generally cannot be spatially focused within the body, and the speed-of-light propagation is considered an instantaneous excitation, which can be considered like a ‘flash’, everywhere, at time=0. The light energy is absorbed to varying degrees in various tissues to create very rapid, localized thermal expansion, which acts as an acoustic source that launches an ultrasonic pressure wave. The resulting ultrasound waves can be detected by a conventional handheld probe with transducer therein, and used to generate an image that is effectively a map of optical absorption within the tissue. In this exemplary embodiment light energy is transmitted into tissue, rather than acoustic energy as in the case of ultrasound imaging. A probe (with transducer therein) used for photoacoustic imaging can thus be used with any of the systems and methods herein, such as by securing an orientation sensor in a fixed position relative to the probe. The embodiments herein are thus not limited to ultrasound transducers being the source of acoustic energy. In FIG. 8, the transmitting arrow from housing 95 to probe 92 is dashed (optional) to reflect embodiments such as photoacoustic imaging, in which laser/light energy is transmitted.

The orientation methods described above, including image annotation and reference icon creation (such as shown in FIGS. 10A, 10B, 11A, 11B, 12A, 12B, 13A, and 13B), are described in the context of methods that receive ultrasound signals from the patient, and then use those received ultrasound signals. Alternatively, the orientation methods herein can conceivably be used with the receipt of forms of energy other than ultrasound. For example, the method herein can be used to orient other imaging modalities such as, for example without limitation, fluoroscopy (x-ray), infrared, or even yet to be discovered forms of imaging using energy transmitted into or emitted from the body. In a particular embodiment, an optical sensor (an optical transmit and receive probe) is an example of a device that could utilize any of the orientation methods herein. The orientation methods for 3D visualization are thus not limited to ultrasound or the systems and device described herein.

### EXAMPLES

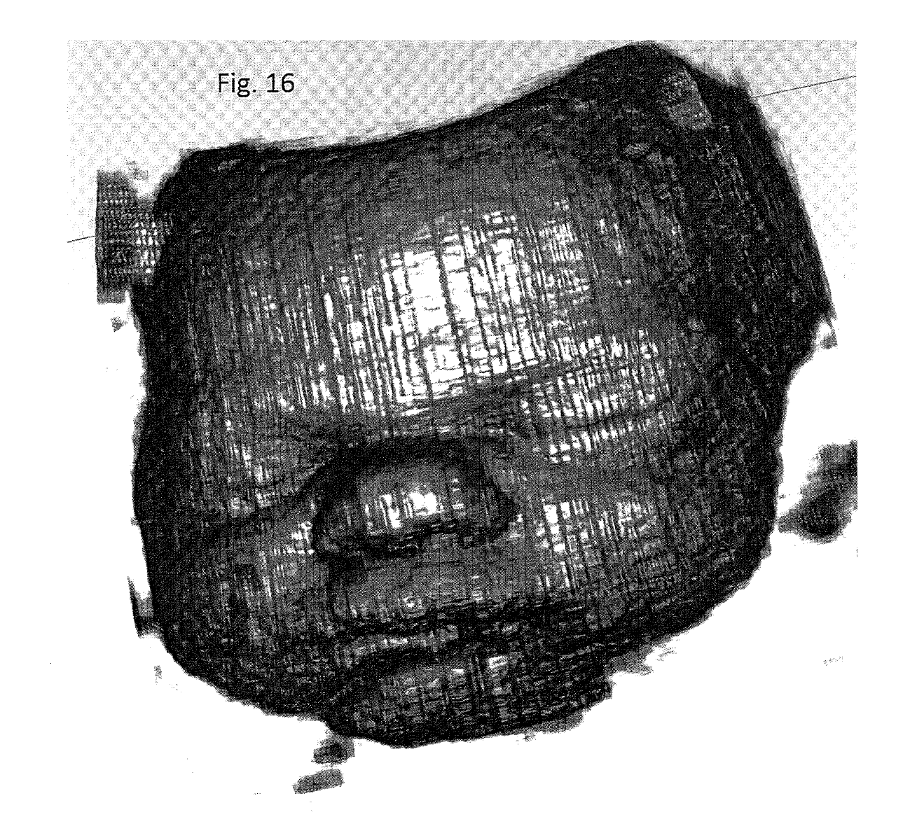

FIG. 16 is a volume-generated 3D image of the face of a 36-week fetal phantom acquired and reconstructed using methods herein and an ultrasound system with only-2D-capable ultrasound scanner and probe. FIG. 16 is thus an example of the step of 3D volume generation herein, and is an example of a 3D image volume generated by devices and/or systems herein. For example, any of the computer executable methods herein that can generate a 3D volume can be used to generate a 3D volume such as that shown in FIG. 16. As set forth above, the devices and systems herein are a fraction of the cost of premium 3D ultrasound scanners and probes currently on the market, yet the 3D image quality is comparable to these expensive, high-end systems.

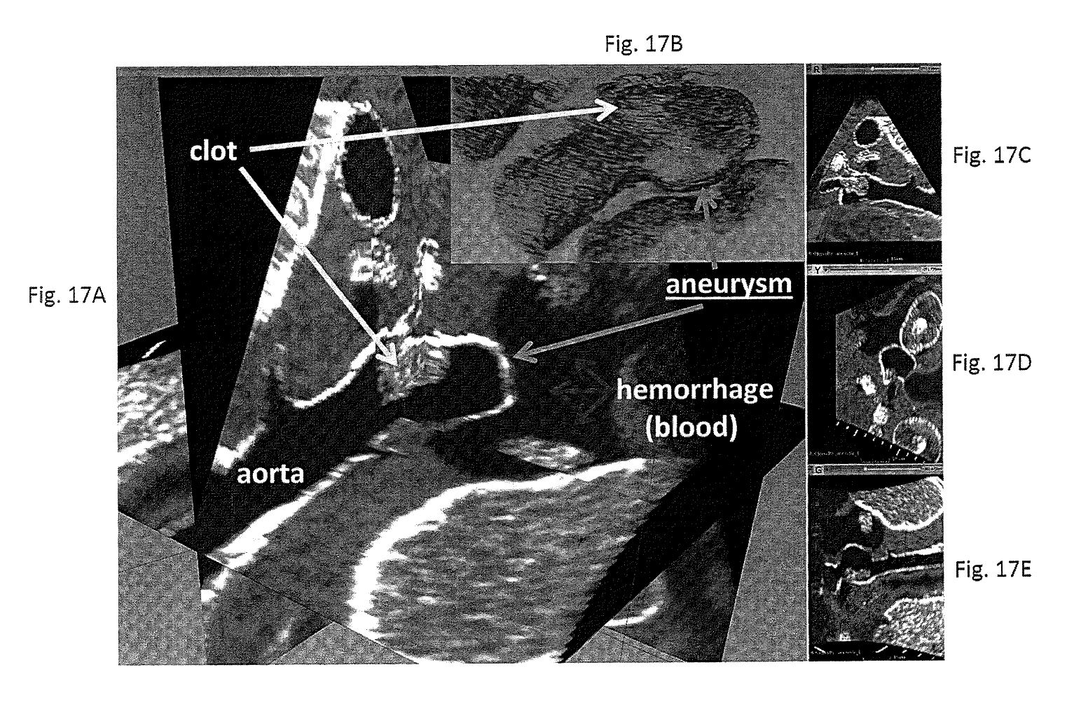

FIGS. 17A-E illustrate visualizations of (i.e., additional images that can be obtained from) a 3D volume generated using systems and methods herein that tag electronic signals with sensed orientation information. These visualizations were created using the software package 3D Slicer to load and manipulate the 3D generated volume, though any 3D medical image data visualization platform (e.g., a DICOM viewer such as OsiriX) may be used for such a task. In this particular embodiment, a portion of the abdominal aorta with a clot, aneurysm, and hemorrhage (as depicted by an ultrasound training simulator) has been acquired and generated as a 3D volume of ultrasound data using systems and methods herein. FIG. 17A illustrates three intersecting 2D cross-sectional planes through the 3D volume of (simulated) ultrasound data obtained and generated using the systems and methods herein, with each 2D cross-sectional image of a plane generally orthogonal to the other two planes, merged together at the relative intersection lines to provide a more detailed spatial illustration of the anatomical region. FIG. 17A indicates the position of a blood clot, hemorrhage, and aneurysm easily identified using the combined 2D ultrasound images. FIG. 17B illustrates a 3D rendering of the same volume of data as in FIG. 17A. The clot and aneurysm are also labeled on the 3D rendered image of the volume. FIGS. 17C, D, E, illustrate the individual 2D ultrasound images which are shown as intersecting in FIG. 17A. Any 3D medical image data visualization platform (e.g., 3D Slicer) can also be loaded onto any of the devices herein (e.g., ultrasound scanner, external device) to allow the 3D volume to be visualized and/or manipulated (during or after 3D volume image generation), such as in the exemplary visualizations in FIGS. 13A-E.

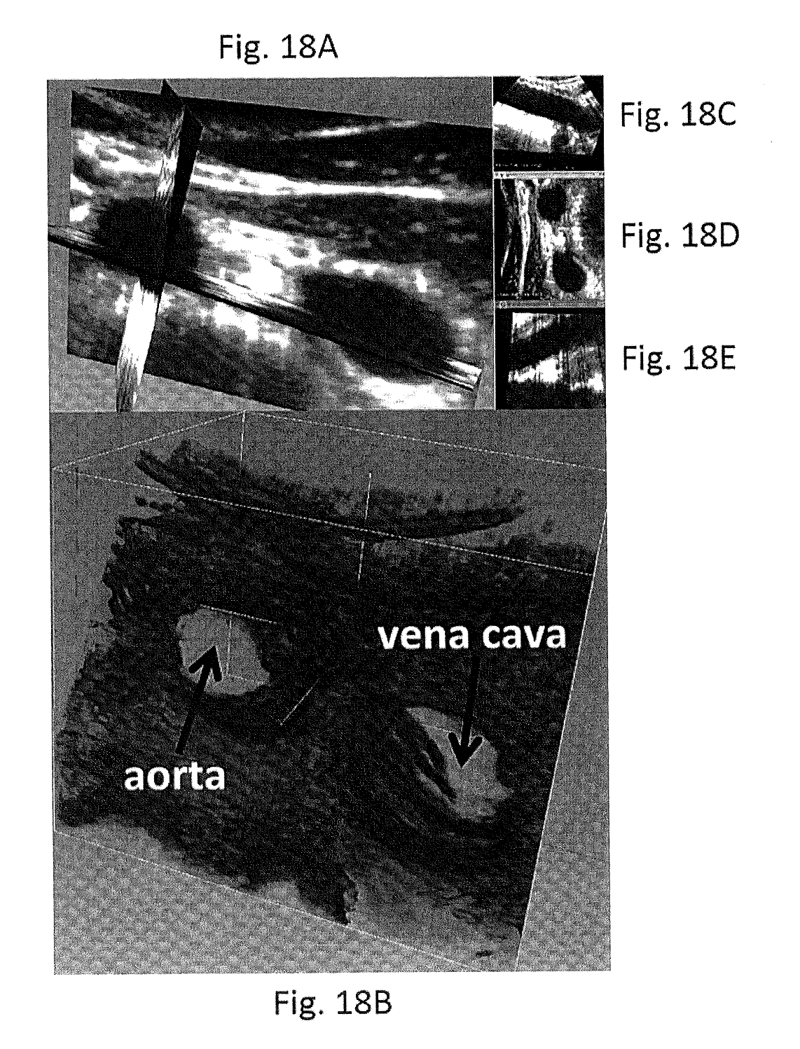

FIGS. 18A-E illustrate the same type of visualizations as in FIGS. 17A-E, but of the aorta and inferior vena cava of a healthy human subject. In this embodiment, the 3D volume of data was generated using systems and methods herein with a clinical ultrasound scanner and probe acquiring 2D images as input, along with the probe-attached sensor readings. The aorta and vena cava are labeled in FIG. 18B.

Arrows with dashed (broken) lines in the figures herein are meant to indicate optional steps.

Any of the methods herein can be used with any suitable device, system, or apparatus herein, and any device, system, or apparatus can be used with any suitable method herein.

1. A method of generating a 3D ultrasound volume image, comprising:
moving an ultrasound transducer and an orientation sensor stabilized with respect to the ultrasound transducer, while restricting the movement of the ultrasound transducer about an axis or point;
tagging each of a plurality of frames of electronic signals indicative of information received by the ultrasound transducer with information sensed by the orientation sensor, relative to the axis or point, each of the plurality of frames of electronic signals indicative of information received by the ultrasound transducer representing a plane or 3D volume of information within the patient; and
generating a 3D ultrasound volume image of the patient by positioning the plurality of tagged frames of electronic signals indicative of information received by the ultrasound transducer at their respective orientations relative to the axis or point.

2. The method of claim 1, wherein the method does not include sensing a position of the transducer with a position sensor.

3. The method of claim 1, wherein tagging each of the plurality of frames of electronic signals indicative of information received by the ultrasound transducer comprises tagging each of a plurality of 2D ultrasound image data with information sensed by the orientation sensor, and wherein generating a 3D ultrasound volume image comprises generating a 3D ultrasound volume image of the patient by positioning the plurality of tagged 2D ultrasound image data at their respective orientations relative to the axis or point.

4. The method of claim 3, wherein tagging each of the plurality of 2D ultrasound image data comprises tagging each of a plurality of detected data.

5. The method of claim 1, wherein tagging each of the plurality of frames of electronic signals indicative of information received by the ultrasound transducer comprises tagging each of a plurality of frames of electronic signals that have not been processed into 2D ultrasound image data.

6. The method of claim 1 further comprising, prior to acquiring the electronic signals indicative of information received by the ultrasound transducer and prior to moving transducer while restricted about the axis or point, calibrating the orientation sensor relative to a patient reference.

7. The method of claim 1 wherein movement is restricted due to an interface between an ultrasound probe and a movement restrictor.

8. The method of claim 7 wherein the interface comprises an interface between at least one surface of the ultrasound probe and a movement restrictor that is positioned on the patient.

9. The method of claim 8 wherein the interface is created by positioning the ultrasound probe within a cradle configured to receive a portion of the probe therein.

10. The method of claim 9, the movement restrictor further configured and adapted such that the cradle can be separately restricted in movement about at least two axes or points.

11. The method of claim 1 wherein the moving step comprises moving an ultrasound probe with the transducer and orientation sensor disposed therein.

12. The method of claim 1 further comprising securing a sensing member comprising the orientation sensor to an ultrasound probe, the probe comprising the transducer.

13. The method of claim 12 wherein securing the sensing member comprises securing the sensing member to a proximal region of the probe, optionally where a cable extends from a probe housing.

14. The method of claim 1 further comprising connecting an external device to a data port on an ultrasound scanner housing, and wherein generating the 3D volume comprises generating the 3D volume on the external device, and displaying the 3D volume on the external device.

15. The method of claim 14 wherein the tagging step occurs on the external device.

16. The method of claim 1 wherein the tagging step occurs in an ultrasound system housing.

17. The method of claim 16 wherein generating a 3D ultrasound volume image also occurs in the ultrasound system housing.

18. The method of claim 1 further comprising storing in memory the plurality of electronic signals indicative of information received by the ultrasound transducer and corresponding information sensed by the orientation sensor.

19. The method of claim 1 further comprising storing in memory the plurality of tagged electronic signals indicative of information received by the ultrasound transducer.

20. The method of claim 1 wherein restricting the movement comprises engaging an ultrasound probe, which comprises the transducer, with a hand of a user.

21. The method of claim 1 wherein the axis or point is a first axis or point, the method further comprising restricting movement of the transducer about a second axis or point, further comprising tagging each of a second plurality of frames of electronic signals indicative of information received by the ultrasound transducer with information sensed by the orientation sensor, relative to the second axis or point, each of the second plurality of electronic signals indicative of information received by the ultrasound transducer; and
generating a second 3D ultrasound volume of the patient by positioning the second plurality of tagged electronic signals indicative of information received by the ultrasound transducer at their respective orientations relative to the second particular axis or point.

22. The method of claim 21, wherein the 3D ultrasound volume image is a first 3D ultrasound volume image, the method further comprising combining at least the first 3D ultrasound volume and the second 3D ultrasound volumes together.

23. The method of claim 22 combining the first and second 3D volumes creates a combined 3D volume with an extended field of view relative to the first and second 3D volumes individually.

24. The method of claim 22 wherein combining the first and second 3D volumes creates a combined 3D volume with improved image quality compared to the first and second 3D volumes individually.

25. The method of claim 21 wherein restricting movement about the first axis or point and the second axis or point is performed using a single movement restrictor.

26. The method of claim 21 wherein restricting movement about the first axis or point is performed with a first movement restrictor, and wherein restricting movement about the second axis or point is performed with a second movement restrictor.

27. The method of claim 26 further comprising securing the first movement restrictor to the second movement restrictor at a known orientation, optionally co-planar, angled, or perpendicular.

28. The method of claim 1 wherein generating a 3D ultrasound volume image of the patient occurs real-time or near-real time with the movement of the ultrasound transducer.

29. The method of claim 1 wherein generating a 3D ultrasound volume image of the patient does not occur real-time or near-real time with the movement of the ultrasound transducer.

30. A computer executable method for tagging frames of electronic signals indicative of information received by an ultrasound transducer, comprising:
receiving as input a plurality of frames of electronic signals indicative of information received by the ultrasound transducer, the plurality of frames of electronic signals representing a plane or 3D volume of information within a patient, wherein the movement of the ultrasound transducer was limited about an axis or point when moved with respect to the patient;
receiving as input information sensed by an orientation sensor stabilized in place with respect to the ultrasound transducer; and
tagging each of the plurality of frames of electronic signals indicative of information received by the ultrasound transducer with information sensed by an orientation sensor.

31. The computer executable method of claim 30 wherein the computer executable method does not include receiving as input position information of the transducer sensed by a position sensor.

32. The computer executable method of claim 30 wherein receiving as input a plurality of frames of electronic signals indicative of information received by the ultrasound transducer comprises receiving as input a plurality of 2D ultrasound image data, and the tagging step comprises tagging each of the plurality of 2D ultrasound data with information sensed by the orientation sensor.

33. The computer executable method of claim 30, wherein the computer executable method is disposed in an ultrasound system housing that includes hardware and software for generating and/or processing ultrasound data.

34. A computer executable method for generating a 3D volume image of a patient, comprising:
receiving as input a plurality of tagged frames of electronic signals indicative of information received by the ultrasound transducer, the plurality of tagged frames of electronic signals each representing a plane or 3D volume of information within a patient, each of the received plurality of frames of electronic signals tagged with information sensed by an orientation sensor stabilized in place with respect to the ultrasound transducer, wherein the movement of the ultrasound transducer was limited about a particular axis or point when moved with respect to the patient; and
generating a 3D ultrasound volume image by positioning the plurality of tagged frames of electronic signals indicative of information received by the ultrasound transducer at their respective orientations relative to the particular axis or point.

35. The computer executable method of claim 34 wherein the computer executable method does not include receiving as input position information of the transducer sensed by a position sensor.

36. The computer executable method of claim 34 wherein receiving as input a plurality of tagged frames of electronic signals indicative of information received by the ultrasound transducer comprises receiving as input a plurality of tagged 2D ultrasound image data, and wherein generating the 3D ultrasound volume comprises positioning the plurality of tagged 2D ultrasound image data at their respective orientations relative to the particular axis or point.

37. A 3D ultrasound image volume generating apparatus, comprising:
an ultrasound probe in a fixed position relative to an orientation sensor;
a movement restrictor configured so as to restrict the movement of the ultrasound probe about a particular axis or point;
a tagging module adapted to tag each of a plurality of frames of electronic signals indicative of information received by the ultrasound transducer with information sensed by the orientation sensor, relative to the particular axis or point; and
a 3D volume generating module adapted to position each of the plurality of orientation tagged frames of electronic signals indicative of information received by the ultrasound transducer at respective orientations, relative to the particular axis or point, to generate a 3D image.

38. The apparatus of claim 37 wherein the movement restrictor is integral with the ultrasound probe.

39. The apparatus of claim 37 wherein the movement restrictor is configured with at least one surface to interface with the ultrasound probe so as to restrict the movement of the ultrasound probe about a particular axis or point.

40. The apparatus of claim 37 wherein the orientation sensor is disposed within a body of the ultrasound probe.

41. The apparatus of claim 37 wherein the orientation sensor is adapted to be removably secured to the ultrasound probe.

42. The apparatus of claim 41 further comprising a sensing member comprising the orientation sensor, the sensing member configured with at least one surface such that it can be secured to a proximal portion of the ultrasound probe, optionally where a probe housing meets a probe cable.

43. The apparatus of claim 42 wherein the sensing member comprises a probe interface, the probe interface having an opening with a greatest linear dimension of 10 mm-35 mm, optionally 15 mm-30 mm.

44. The apparatus of claim 37, not comprising a position sensor.

45. The apparatus of claim 37 wherein the movement restrictor comprises an axis or point selector adapted so that the movement restrictor can restrict the movement of the ultrasound probe about a second axis or point.

46. The apparatus of claim 37 wherein the movement restrictor is configured with at least one surface such that it can be positioned on the body of a patient.

47. The apparatus of claim 37 further comprising an external device in communication with an ultrasound system, the external device comprising the tagging module, and receiving as input the plurality of frames of electronic signals indicative of information received by the ultrasound transducer.

48. The apparatus of claim 47, the external device also in communication with the orientation sensor.

49. The apparatus of claim 47, the external device further comprising the 3D volume generating module.

50. The apparatus of claim 47 wherein the external device is in communication with a video out port of the ultrasound system.

51. The apparatus of claim 47 wherein the external device is in communication with the ultrasound system to enable the external device to receive as input from the ultrasound system at least one of raw channel data, raw beamformed data, and detected data.

52. The apparatus of claim 37 further comprising a second movement restrictor configured to be stabilized with respect to the movement restrictor, the second movement restrictor configured with at least one surface to interface with the ultrasound probe so as to restrict the movement of the ultrasound probe about a second particular axis or point.

53. The apparatus of claim 52 wherein the tagging module is adapted to tag each of a plurality of frames of electronic signals indicative of information received by the ultrasound transducer with information sensed by the orientation sensor, relative to the second particular axis or point, wherein the 3D volume generating module is adapted to generate a second 3D ultrasound volume of the patient by positioning the second plurality of tagged frames of electronic signals indicative of information received by the ultrasound transducer at their respective orientations relative to the second particular axis or point.

54. The apparatus of claim 53 wherein the 3D volume generating module is adapted to merge the 3D ultrasound volume and the second 3D ultrasound volume together.

55. The apparatus of claim 37 wherein the tagging module and the 3D volume generating module are disposed within an ultrasound system housing.

56. An ultrasound imaging apparatus, comprising:
an ultrasound probe in a fixed position relative to an orientation sensor; and
a movement restrictor configured with at least one surface to interface with the ultrasound probe, and adapted so as to limit the movement of the ultrasound probe about an axis or point, the movement restrictor further comprising at least one surface adapted to interface with the body of a patient.

57. The ultrasound imaging apparatus of claim 56, wherein the movement restrictor has at least a first configuration and a second configuration, wherein the first configuration restricts the ultrasound probe's movement about the axis or point, and the second configuration restricts the ultrasound probe's movement about a second axis or point.

58. The ultrasound imaging apparatus of claim 56 wherein the movement restrictor is adapted and configured to limit the movement of the probe about a first axis, the movement restrictor further adapted and configured to limit the movement of the probe about a second axis, the first and second axes being in the same plane.

59. The apparatus of claim 56 wherein the movement restrictor comprises a probe cradle with at least one surface to interface with the ultrasound probe.

60. The apparatus of claim 56 wherein the movement restrictor further comprises an axis selector, which is adapted to be moved to select one of at least two axes or points for restriction of movement.

61. The apparatus of claim 56 further comprising a second movement restrictor configured to stably interface with the movement restrictor, the second movement restrictor adapted to so as to limit the movement of the ultrasound probe about a second axis or point.

62. A 3D ultrasound volume generating system, comprising:
a freehand ultrasound transducer in a fixed position relative to an orientation sensor, and not a position sensor,
the system adapted to generate a 3D ultrasound volume using sensed information provided from the orientation sensor that is tagged to frames of electronic signals indicative of information received by the ultrasound transducer, and without information sensed from a position sensor.

63. The system of claim 62 further comprising a probe movement restrictor with at least one surface configured to interface with an ultrasound probe, to limit the movement of the ultrasound transducer about an axis or point.

64. A method of generating a 3D ultrasound image volume, comprising:
scanning a patient's body with an ultrasound probe in a fixed position relative to an orientation sensor;
sensing orientation information while moving the probe, but not sensing x-y-z position information of the probe; and
generating a 3D ultrasound volume from a plurality of frames of electronic signals indicative of information received by the ultrasound transducer.

65. The method of claim 64 further comprising restricting the movement of the probe about an axis or point.

66. A sensing member with at least one surface configured to be removably secured in a fixed position relative to an ultrasound probe, the sensing member comprising an orientation sensor and not a position sensor.

67. The sensing member of claim 66 wherein the sensing member has an opening with a largest linear dimension from 10 mm-35 mm, optionally 15 mm-30 mm.

68. The sensing member of claim 66 wherein the sensing member comprises a deformable element configured to be deformed to allow the sensing member to be secured to the ultrasound probe.
